(19) United States
(12) Patent Application Publication
(10) Pub. No.: US 2009/0226455 A1
(43) Pub. Date: Sep. 10, 2009

(54) COMBINATION THERAPY WITH C-MET AND HER ANTAGONISTS

(75) Inventor: Ellen Filvaroff, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/399,879

(22) Filed: Mar. 6, 2009

Related U.S. Application Data
(60) Provisional application No. 61/044,433, filed on Apr. 11, 2008.
(60) Provisional application No. 61/034,453, filed on Mar. 6, 2008.

(51) Int. Cl. A61K 39/395; A61P 35/00

(52) U.S. Cl. 424/139.1

(57) ABSTRACT

The present invention relates generally to the fields of molecular biology and growth factor regulation. More specifically, the invention relates to combination therapies for the treatment of pathological conditions, such as cancer.

### CROSS-REFERENCE TO RELATED APPLICATIONS

This application claims priority under 35 USC §119(e) to U.S. provisional application No. 61/034,453, filed Mar. 6, 2008, and U.S. provisional application No. 61/044,433, filed Apr. 11, 2008, the contents of which are incorporated herein by reference.

### TECHNICAL FIELD

The present invention relates generally to the fields of molecular biology and growth factor regulation. More specifically, the invention relates to combination therapies for the treatment of pathological conditions, such as cancer.

### BACKGROUND

HGF is a mesenchyme-derived pleiotrophic factor with mitogenic, motogenic and morphogenic activities on a number of different cell types. HGF effects are mediated through a specific tyrosine kinase, c-met, and aberrant HGF and c-met expression are frequently observed in a variety of tumors. See, e.g., Maulik et al., Cytokine & Growth Factor Reviews (2002), 13:41-59; Danilkovitch-Miagkova & Zbar, J. Clin. Invest. (2002), 109(7):863-867. Regulation of the HGF/c-Met signaling pathway is implicated in tumor progression and metastasis. See, e.g., Trusolino & Comoglio, Nature Rev. (2002), 2:289-300).

HGF binds the extracellular domain of the Met receptor tyrosine kinase (RTK) and regulates diverse biological processes such as cell scattering, proliferation, and survival. HGF-Met signaling is essential for normal embryonic development especially in migration of muscle progenitor cells and development of the liver and nervous system (Bladt et al., Nature (1995), 376, 768-771.; Hamanoue et al., Faseb J (2000), 14, 399-406; Maina et al., Cell (1996), 87, 531-542; Schmidt et al., Nature (1995), 373, 699-702; Uehara et al., Nature (1995), 373, 702-705). Developmental phenotypes of Met and HGF knockout mice are very similar suggesting that HGF is the cognate ligand for the Met receptor (Schmidt et al., 1995, supra; Uehara et al., 1995, supra). HGF-Met also plays a role in liver regeneration, angiogenesis, and wound healing (Bussolino et al., J Cell Biol (1992), 119, 629-641; Matsumoto and Nakamura, Exs (1993), 65, 225-249; Nusrat et al., J Clin Invest (1994) 93, 2056-2065). The precursor Met receptor undergoes proteolytic cleavage into an extracellular α subunit and membrane spanning β subunit linked by disulfide bonds (Tempest et al., Br J Cancer (1988), 58, 3-7). The β subunit contains the cytoplasmic kinase domain and harbors a multi-substrate docking site at the C-terminus where adapter proteins bind and initiate signaling (Bardelli et al., Oncogene (1997), 15, 3103-3111; Nguyen et al., J Biol Chem (1997), 272, 20811-20819; Pelicci et al., Oncogene (1995), 10, 1631-1638; Ponzetto et al., Cell (1994), 77, 261-271; Weidner et al., Nature (1996), 384, 173-176). Upon HGF binding, activation of Met leads to tyrosine phosphorylation and downstream signaling through Gab1 and Grb2/Sos mediated PI3-kinase and Ras/MAPK activation respectively, which drives cell motility and proliferation (Furge et al., Oncogene (2000), 19, 5582-5589; Hartmann et al., J Biol Chem (1994), 269, 21936-21939; Ponzetto et al., J Biol Chem (1996), 271, 14119-14123; Royal and Park, J Biol Chem (1995), 270, 27780-27787).

Met was shown to be transforming in a carcinogen-treated osteosarcoma cell line (Cooper et al, Nature (1984), 311, 29-33; Park et al., Cell (1986), 45, 895-904). Met overexpression or gene-amplification has been observed in a variety of human cancers. For example, Met protein is overexpressed at least 5-fold in colorectal cancers and reported to be gene-amplified in liver metastasis (Di Renzo et al., Clin Cancer Res (1995), 1, 147-154; Liu et al., Oncogene (1992), 7, 181-185). Met protein is also reported to be overexpressed in oral squamous cell carcinoma, hepatocellular carcinoma, renal cell carcinoma, breast carcinoma, and lung carcinoma (Jin et al., Cancer (1997), 79, 749-760; Morello et al., J Cell Physiol (2001), 189, 285-290; Natali et al., Int J Cancer (1996), 69, 212-217; Olivero et al., Br J Cancer (1996), 74, 1862-1868; Suzuki et al., Br J Cancer (1996), 74, 1862-1868). In addition, overexpression of mRNA has been observed in hepatocellular carcinoma, gastric carcinoma, and colorectal carcinoma (Boix et al., Hepatology (1994), 19, 88-91; Kuniyasu et al., Int J Cancer (1993), 55, 72-75; Liu et al., Oncogene (1992), 7, 181-185).

A number of mutations in the kinase domain of Met have been found in renal papillary carcinoma which leads to constitutive receptor activation (Olivero et al., Int J Cancer (1999), 82, 640-643; Schmidt et al., Nat Genet (1997), 16, 68-73; Schmidt et al., Oncogene (1999), 18, 2343-2350). These activating mutations confer constitutive Met tyrosine phosphorylation and result in MAPK activation, focus formation, and tumorigenesis (Jeffers et al., Proc Natl Acad Sci U S A (1997), 94, 11445-11450). In addition, these mutations enhance cell motility and invasion (Giordano et al., Faseb J (2000), 14, 399-406; Lorenzato et al., Cancer Res (2002), 62, 7025-7030). HGF-dependent Met activation in transformed cells mediates increased motility, scattering, and migration which eventually leads to invasive tumor growth and metastasis (Jeffers et al., Mol Cell Biol (1996), 16, 1115-1125; Meiners et al., Oncogene (1998), 16, 9-20).

Met has been shown to interact with other proteins that drive receptor activation, transformation, and invasion. In neoplastic cells, Met is reported to interact with α6β4 integrin, a receptor for extracellular matrix (ECM) components such as laminins, to promote HGF-dependent invasive growth (Trusolino et al., Cell (2001), 107, 643-654). In addition, the extracellular domain of Met has been shown to interact with a member of the semaphorin family, plexin B1, and to enhance invasive growth (Giordano et al., Nat Cell Biol (2002), 4, 720-724). Furthermore, CD44v6, which has been implicated in tumorigenesis and metastasis, is also reported to form a complex with Met and HGF and result in Met receptor activation (Orian-Rousseau et al., Genes Dev (2002), 16, 3074-3086).

Met is a member of the subfamily of receptor tyrosine kinases (RTKs) which include Ron and Sea (Maulik et al., Cytokine Growth Factor Rev (2002), 13, 41-59). Prediction of the extracellular domain structure of Met suggests shared homology with the semaphorins and plexins. The N-terminus of Met contains a Sema domain of approximately 500 amino acids that is conserved in all semaphorins and plexins. The semaphorins and plexins belong to a large family of secreted and membrane-bound proteins first described for their role in neural development (Van Vactor and Lorenz, Curr Bio (1999), 19, R201-204). However, more recently semaphorin overexpression has been correlated with tumor invasion and metastasis. A cysteine-rich PSI domain (also referred to as a Met Related Sequence domain) found in plexins, semaphorins, and integrins lies adjacent to the Sema domain followed by four IPT repeats that are immunoglobulin-like regions found in plexins and transcription factors. A recent study suggests that the Met Sema domain is sufficient for HGF and heparin binding (Gherardi et al., Proc Natl Acad Sci USA (2003), 100(21):12039-44).

As noted above, the Met receptor tyrosine kinase is activated by its cognate ligand HGF and receptor phosphorylation activates downstream pathways of MAPK, PI-3 kinase and PLC-γ (L. Trusolino and P. M. Comoglio, Nat Rev Cancer 2, 289 (2002); C. Birchmeier et al., Nat Rev Mol Cell Biol 4, 915 (2003)). Phosphorylation of Y1234/Y1235 within the kinase domain is critical for Met kinase activation while Y1349 and Y1356 in the multisubstrate docking site are important for binding of src homology-2 (SH2), phosphotyrosine binding (PTB), and Met binding domain (MBD) proteins (C. Ponzetto et al., Cell 77, 261 (1994); K. M. Weidner et al., Nature 384, 173 (1996); G. Pelicci et al., Oncogene 10, 1631 (1995)) to mediate activation of downstream signaling pathways. An additional juxtamembrane phosphorylation site, Y1003, has been well characterized for its binding to the tyrosine kinase binding (TKB) domain of the Cbl E3-ligase (P. Peschard et al., Mol Cell 8, 995 (2001); P. Peschard, N. Ishiyama, T. Lin, S. Lipkowitz, M. Park, J Biol Chem 279, 29565 (2004)). Cbl binding is reported to drive endophilin-mediated receptor endocytosis, ubiquitination, and subsequent receptor degradation (A. Petrelli et al., Nature 416, 187 (2002)). This mechanism of receptor downregulation has been described previously in the EGFR family that also harbor a similar Cbl binding site (K. Shtiegman, Y. Yarden, Semin Cancer Biol 13, 29 (2003); M. D. Marmor, Y. Yarden, Oncogene 23, 2057 (2004); P. Peschard, M. Park, Cancer Cell 3, 519 (2003)). Dysregulation of Met and HGF have been reported in a variety of tumors. Ligand-driven Met activation has been observed in several cancers. Elevated serum and intra-tumoral HGF is observed in lung, breast cancer, and multiple myeloma (J. M. Siegfried et al., Ann Thorac Surg 66, 1915 (1998); P. C. Ma et al., Anticancer Res 23, 49 (2003); B. E. Elliott et al. Can J Physiol Pharmacol 80, 91 (2002); C. Seidel, et al., Med Oncol 15, 145 (1998)). Overexpression of Met and/or HGF, Met amplification or mutation has been reported in various cancers such as colorectal, lung, gastric, and kidney cancer and is thought to drive ligand-independent receptor activation (C. Birchmeier et al., Nat Rev Mol Cell Biol 4, 915 (2003); G. Maulik et al., Cytokine Growth Factor Rev 13, 41 (2002)). Additionally, inducible overexpression of Met in a liver mouse model gives rise to hepatocellular carcinoma demonstrating that receptor overexpression drives ligand independent tumorigenesis (R. Wang, et al., J Cell Biol 153, 1023 (2001)). The most compelling evidence implicating Met in cancer is reported in familial and sporadic renal papillary carcinoma (RPC) patients. Mutations in the kinase domain of Met that lead to constitutive activation of the receptor were identified as germline and somatic mutations in RPC (L. Schmidt et al., Nat Genet 16, 68 (1997)). Introduction of these mutations in transgenic mouse models leads to tumorigenesis and metastasis. (M. Jeffers et al., Proc Natl Acad Sci USA 94, 11445 (1997)).

Publications relating to c-met and c-met antagonists include Martens, T, et al. (2006) Clin Cancer Res 12(20 Pt 1):6144; U.S. Pat. No. 6,468,529; WO2006/015371; WO2007/063816; WO2006/104912; WO2006/104911; WO2006/113767; US2006-0270594; US2006-0270594; US2006-0293235; US Pat. No. 7,481,993; WO2009/007427; WO2005/016382; WO2009/002521; WO2007/143098; WO2007/115049; WO2007/126799. Combination therapies with met antagonist and EGFR antagonists are described in co-owned, co-pending U.S. patent application Ser. No. ______, filed Mar. 6, 2009, U.S. provisional application No. 61/034,446, filed Mar. 6, 2008, and U.S. provisional application No. 61/044,438, filed Apr. 11, 2008.

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185neu), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway. Baselga and Mendelsohn Pharmac. Ther. 64:127-154 (1994). Monoclonal antibodies directed against the EGFR or its ligands, TGF-α and EGF, have been evaluated as therapeutic agents in the treatment of such malignancies. See, e.g., Baselga and Mendelsohn., supra; Masui et al. Cancer Research 44:1002-1007 (1984); and Wu et al. J. Clin. Invest. 95:1897-1905 (1995).

The second member of the HER family, p 185^{neu}, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Slamon et al., Science, 244:707-712 (1989); and U.S. Pat. No. 4,968,603). To date, no point mutation analogous to that in the neu proto-oncogene has been reported for human tumors. Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al., Mol. Carcinog., 3:254-257 (1990); Aasland et al. Br. J. Cancer 57:358-363 (1988); Williams et al. Pathobiology 59:46-52 (1991); and McCann et al., Cancer, 65:88-92 (1990). HER2 maybe overexpressed in prostate cancer (Gu et al. Cancer Lett. 99:185-9 (1996); Ross et al. Hum. Pathol. 28:827-33 (1997); Ross et al. Cancer 79:2162-70 (1997); and Sadasivan et al. J. Urol. 150:126-31 (1993)).

Antibodies directed against the rat p185^{neu} and human HER2 protein products have been described.

Drebin and colleagues have raised antibodies against the rat neu gene product, p185neu See, for example, Drebin et al., Cell 41:695-706 (1985); Myers et al., Meth. Enzym. 198:277-290 (1991); and WO94/22478. Drebin et al. Oncogene 2:273-277 (1988) report that mixtures of antibodies reactive with two distinct regions of p185^{neu} result in synergistic anti-tumor effects on neu-transformed NIH-3T3 cells implanted into nude mice. See also U.S. Pat. No. 5,824,311 issued Oct. 20, 1998.

Hudziak et al., Mol. Cell. Biol. 9(3):1165-1172 (1989) describe the generation of a panel of HER2 antibodies which were characterized using the human breast tumor cell line SK-BR-3. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-overexpressing breast tumor cell lines to the cytotoxic effects of TNF-α. See also U.S. Pat. No. 5,677,171 issued Oct. 14, 1997. The HER2 antibodies discussed in Hudziak et al. are further characterized in Fendly et al. Cancer Research 50:1550-1558 (1990); Kotts et al. In Vitro 26(3):59A (1990); Sarup et al. Growth Regulation 1:72-82 (1991); Shepard et al. J. Clin. Immunol. 11(3):117-127 (1991); Kumar et al. Mol. Cell. Biol. 11(2):979-986 (1991); Lewis et al. Cancer Immunol. Immunother. 37:255-263 (1993); Pietras et al. Oncogene 9:1829-1838 (1994); Vitetta et al. Cancer Research 54:5301-5309 (1994); Sliwkowski et al. J. Biol Chem. 269(20):14661-14665 (1994); Scott et al. J. Biol. Chem. 266:14300-5 (1991); D'souza et al. Proc. Natl. Acad. Sci. 91:7202-7206 (1994); Lewis et al. Cancer Research 56:1457-1465 (1996); and Schaefer et al. Oncogene 15:1385-1394 (1997).

A recombinant humanized version of the murine HER2 antibody 4D5 (huMAb4D5-8, rhuMAb HER2, trastuzumab or HERCEPTIN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)). Trastuzumab received marketing approval from the Food and Drug Administration Sep. 25, 1998 for the treatment of patients with metastatic breast cancer whose tumors overexpress the HER2 protein.

To target the HER signaling pathway, rhuMAb 2C4 (Pertuzumab) was developed as a humanized antibody that inhibits the dimerization of HER2 with other HER receptors, thereby inhibiting ligand-driven phosphorylation and activation, and downstream activation of the RAS and AKT pathways. In a phase I trial of Pertuzumab as a single agent for treating solid tumors, 3 subjects with advanced ovarian cancer were treated with pertuzumab. One had a durable partial response, and an additional subject had stable disease for 15 weeks. Agus et al. Proc Am Soc Clin Oncol 22: 192, Abstract 771 (2003).

Other HER2 antibodies with various properties have been described in Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; and Klapper et al. Oncogene 14:2099-2109 (1997).

Homology screening has resulted in the identification of two other HER receptor family members; HER3 (U.S. Pat. Nos. 5,183,884 and 5,480,968 as well as Kraus et al. PNAS (USA) 86:9193-9197 (1989)) and HER4 (EP Pat Appln No 599,274; Plowman et al., Proc. Natl. Acad. Sci. USA, 90:1746-1750 (1993); and Plowman et al., Nature, 366:473-475 (1993)). Both of these receptors display increased expression on at least some breast cancer cell lines.

The HER receptors are generally found in various combinations in cells and heterodimerization is thought to increase the diversity of cellular responses to a variety of HER ligands (Earp et al. Breast Cancer Research and Treatment 35: 115-132 (1995)). EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors 11:235-257 (1994)). A family of heregulin proteins resulting from alternative splicing of a single gene are ligands for HER3 and HER4. The heregulin family includes alpha, beta and gamma heregulins (Holmes et al., Science, 256:1205-1210 (1992); U.S. Pat. No. 5,641,869; and Schaefer et al. Oncogene 15:1385-1394 (1997)); neu differentiation factors (NDFs), glial growth factors (GGFs); acetylcholine receptor inducing activity (ARIA); and sensory and motor neuron derived factor (SMDF). For a review, see Groenen et al. Growth Factors 11:235-257 (1994); Lemke, G. Molec. & Cell. Neurosci. 7:247-262 (1996) and Lee et al. Pharm. Rev. 47:51-85 (1995). Recently three additional HER ligands were identified; neuregulin-2 (NRG-2) which is reported to bind either HER3 or HER4 (Chang et al. Nature 387 509-512 (1997); and Carraway et al Nature 387:512-516 (1997)); neuregulin-3 which binds HER4 (Zhang et al. PNAS (USA) 94(18):9562-7 (1997)); and neuregulin-4 which binds HER4 (Harari et al. Oncogene 18:2681-89 (1999)) HB-EGF, betacellulin and epiregulin also bind to HER4.

While EGF and TGFα do not bind HER2, EGF stimulates EGFR and HER2 to form a heterodimer, which activates EGFR and results in transphosphorylation of HER2 in the heterodimer. Dimerization and/or transphosphorylation appears to activate the HER2 tyrosine kinase. See Earp et al., supra. Likewise, when HER3 is co-expressed with HER2, an active signaling complex is formed and antibodies directed against HER2 are capable of disrupting this complex (Sliwkowski et al., J. Biol. Chem., 269(20):14661-14665 (1994)). Additionally, the affinity of HER3 for heregulin (HRG) is increased to a higher affinity state when co-expressed with HER2. See also, Levi et al., Journal of Neuroscience 15: 1329-1340 (1995); Morrissey et al., Proc. Natl. Acad. Sci. USA 92: 1431-1435 (1995); and Lewis et al., Cancer Res., 56:1457-1465 (1996) with respect to the HER2-HER3 protein complex. HER4, like HER3, forms an active signaling complex with HER2 (Carraway and Cantley, Cell 78:5-8 (1994)).

Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192211A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,127,526, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2004/0037823A1, US2005/0002928A1, US 6,573,043, U.S. Pat. No. 6,905,830, US2003/0152987A1, WO99/48527, US2002/0141993A1, US2005/0244417A1, U.S. Pat. No. 6,949,245, US2003/0086924, US2004/0013667A1, WO00/69460, US2003/0170235A1, U.S. Pat. No. 7,041,292, WO01/00238, US2006/0083739, WO01/15730, U.S. Pat. No. 6,627,196B1, U.S. Pat. No. 6,632,979B1, WO01/00244, US2002/0001587A1, US2002/0090662A1, U.S. Pat. No. 6,984,494B2, WO01/89566, US2002/0064785, US2003/0134344, WO 2005/099756, US2006/0013819, WO2006/07398A1, US2006/0018899, WO 2006/33700, US2006/0088523, US 2006/0034840, WO 04/24866, US2004/0082047, US2003/0175845A1, WO03/087131, US2003/0228663, WO2004/008099A2, US2004/0106161, WO2004/048525, US2004/0258685A1, WO 2005/16968, US2005/0038231A1 U.S. Pat. No. 5,985,553, U.S. Pat. No. 5,747,261, U.S. Pat. No. 4,935,341, U.S. Pat. No. 5,401,638, U.S. Pat No. 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, US 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, US 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

Despite the significant advancement in the treatment of cancer, improved therapies are still being sought.

All references cited herein, including patent applications and publications, are incorporated by reference in their entirety.

### SUMMARY OF THE INVENTION

The present invention provides combination therapies for treating a pathological condition, such as cancer, wherein a c-Met antagonist is combined with an HER antagonist, thereby providing significant anti-tumor activity.

In one aspect, the invention provides methods of treating a cancer in a subject, comprising administering to the subject a therapeutically effective amount of a c-met antagonist and an HER antagonist.

Examples of c-met antagonists include, but are not limited to, soluble c-met receptors, soluble HGF variants, apatmers or peptibodies that are specific to c-met or HGF, c-met small molecules, anti-c-met antibodies and anti-HGF antibodies. In some embodiment, the c-met antagonist is an anti-c-met antibody.

In one embodiment, the anti-c-met antibody comprises a heavy chain variable domain comprising one or more of CDR1-HC, CDR2-HC and CDR3-HC sequence depicted in FIG. 12 (SEQ ID NOs: 29-31). In some embodiments, the antibody comprises a light chain variable domain comprising one or more of CDR1-LC, CDR2-LC and CDR3-LC sequence depicted in FIG. 12 (SEQ ID NOs: 11, 12, 23). In some embodiments, the heavy chain variable domain comprises FR1-HC, FR2-HC, FR3-HC and FR4-HC sequence depicted in FIG. 12 (SEQ ID NOs: 25-28). In some embodiments, the light chain variable domain comprises FR1-LC, FR2-LC, FR3-LC and FR4-LC sequence depicted in FIG. 12 (SEQ ID NOs: 7-10). In some embodiments, the anti-cmet antibody is monovalent and comprises an Fc region. In some embodiments, the antibody comprises Fc sequence depicted in FIG. 12 (SEQ ID NO:33).

In some embodiments, the antibody is monovalent and comprises a Fc region, wherein the Fc region comprises a first and a second polypeptide, wherein the first polypeptide comprises the Fc sequence depicted in FIG. 12 (SEQ ID NO: 33) and the second polypeptide comprises the Fc sequence depicted in FIG. 13 (SEQ ID NO: 34).

In some embodiments, the anti-c-met antibody comprises (a) a first polypeptide comprising a heavy chain variable domain having the sequence:
QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYVSPLDYWGQGTSVTVSS (SEQ ID NO:35), CH1 sequence depicted in FIG. 12 (SEQ ID NO:32), and the Fc sequence depicted in FIG. 12 (SEQ ID NO:33); and (b) a second polypeptide comprising a light chain variable domain having the sequence:
DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ ID NO:36), and CL1 sequence depicted in FIG. 12 (SEQ ID NO:24); and (c) a third polypeptide comprising the Fc sequence depicted in FIG. 13 (SEQ ID NO:34).

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816. For example, a hole mutation can be one or more of T366A, L368A and/or Y407V in an Fc polypeptide, and a cavity mutation can be T366W in an Fc polypeptide.

In some embodiments, the c-met antagonist is SGX-523, PF-02341066, JNJ-38877605, BMS-698769, PHA-665,752, SU5416, SU 1274, XL-880, MGCD265, ARQ 197, MP-470, AMG 102, antibody 223C4 or humanized antibody 223C4 (WO2009/007427), L2G7, NK4, XL-184, MP-470, or Comp-1.

C-met antagonists can be used to reduce or inhibit one or more aspects of HGF/c-met-associated effects, including but not limited to c-met activation, downstream molecular signaling (e.g., mitogen activated protein kinase (MAPK) phosphorylation, AKT phosphorylation, c-met phosphorylation, PI3 kinase mediated signaling), cell proliferation, cell migration, cell survival, cell morphogenesis and angiogenesis. These effects can be modulated by any biologically relevant mechanism, including disruption of ligand (e.g., HGF) binding to c-met, c-met phosphorylation and/or c-met multimerization.

In some embodiments, the HER antagonist is a HER2 and/or HER3 antagonist. In some embodiments, the HER antagonist is a HER2 antagonist. In other embodiments, the antagonist is a HER3 antagonist. In some embodiments, the HER2 antagonist binds to HER2. In some embodiments, the HER3 antagonist binds to HER3. In some embodiments, the HER antagonist is a HER dimerization inhibitor (e.g., pertuzumab). In some embodiments, the HER antagonist is an antibody (e.g., pertuzumab or trastuzumab). In a particular embodiment, the antibody binds to HER2, such as to Domain II of HER2 extracellular domain, or to a junction between domains I, II and III of HER2 extracellular domain.

HER2 antagonists can be used to reduce or inhibit one or more aspects of HER2-HER2 ligand-associated effects, including but not limited to HER2 activation, downstream molecular signaling, cell proliferation. These effects can be modulated by any biologically relevant mechanism, including disruption of ligand binding to HER2 and disruption of HER2 dimerization.

HER3 antagonists can be used to reduce or inhibit one or more aspects of HER3-HER3 ligand-associated effects, including but not limited to HER3 activation, downstream molecular signaling, cell proliferation. These effects can be modulated by any biologically relevant mechanism, including disruption of ligand binding to HER3, and disruption of HER3 phosphorylation.

Methods of the invention can be used to affect any suitable pathological state. For example, methods of the invention can be used for treating different cancers, both solid tumors and soft-tissue tumors alike. Non-limiting examples of cancers amendable to the treatment of the invention include breast cancer, colorectal cancer, sarcoma, renal cell carcinoma, rectal cancer, non-small cell lung cancer, non-Hodgkins lymphoma (NHL), renal cell cancer, prostate cancer, liver cancer, pancreatic cancer, soft-tissue sarcoma, glioblastoma, kaposi's sarcoma, carcinoid carcinoma, head and neck cancer, melanoma, ovarian cancer, gastric cancer, mesothelioma, and multiple myeloma. In certain aspects, the cancers are metastatic. In other aspects, the cancers are non-metastatic.

In certain embodiments, the cancer is not an EGFR antagonist (e.g., erlotinib or gefitinib) resistant cancer. In certain embodiments, the cancer is not an erlotinib or gefitinib resistant cancer.

In certain embodiments, the cancer is not a tyrosine kinase inhibitor-resistant cancer. In certain embodiments, the cancer is not a small molecule EGFR tyrosine kinase inhibitor-resistant cancer.

In certain embodiments, the cancer displays a wildtype EGFR gene. In certain embodiments, the cancer displays a wildtype EGFR gene and c-met amplification and/or c-met mutation.

In certain embodiments, the cancer displays EGFR mutation. Mutations can be located in any portion of an EGFR gene or regulatory region associated with an EGFR gene. Exemplary EGFR mutations include, for example, mutations in exon 18, 19, 20 or 21, mutations in the kinase domain, G719A, L858R, E746K, L747S, E749Q, A750P, A755V, V765M, S7681, L858P, E746-R748 del, R748-P753 del, M766-A767 A1 ins, S768- V769 SVA ins, P772-H773 NS ins, 2402OC, 2482OA, 2486T>C, 2491 G>C, 2494OC, 251 0OT, 2539OA, 2549OT, 2563OT, 2819T>C, 2482-2490 del, 2486-2503 del, 2544-2545 ins GCCATA, 2554-2555 ins CCAGCGTGG, or 2562-2563 ins AACTCC. Other examples of EGFR activating mutations are known in the art (see e.g., US Patent Publication No. 2005/0272083). In certain embodiments, the cell or cell line does not comprise a T790M mutation in the EGFR gene.

In certain embodiments, the cancer displays c-met and/or HER expression, amplification, or activation. In certain embodiments, the cancer does not display c-met and/or HER expression, amplification, or activation.

In certain embodiments, the cancer displays c-met and/or HER activation. In certain embodiments, the constitutively activated c-met or HER comprises a mutation in the tyrosine kinase domain. In certain embodiments, the cancer does not display c-met and/or HER activation.

In certain embodiments, the cancer displays constitutive c-met and/or HER activation. In some embodiments, the constitutive HER comprises a mutation in the tyrosine kinase domain. In certain embodiments, the cancer does not display constitutive c-met and/or HER activation.

In certain embodiments, the cancer displays ligand-independent c-met and/or HER activation. In certain embodiments, the cancer does not display ligand-independent c-met and/or HER activation.

In certain embodiments, the cancer does not display c-met and/or HER amplification.

In certain embodiments, the cancer displays c-met and/or HER amplification.

In one aspect, the invention provides methods for treating a subject suffering from a cancer that is resistant to treatment with an ErbB antagonist, comprising administering to the subject an ErbB antagonist and a c-met antagonist.

In some embodiments, the cancer is lung cancer, brain cancer, breast cancer, head and neck cancer, colon cancer, ovarian cancer, gastric cancer, or pancreatic cancer. In some embodiments, the cancer is non-small cell lung cancer (NSCLC). In some embodiments, the subject has an EGFR, HER2, HER3, or HER4 activating mutation or gene amplification. In some embodiments, the subject has an EGFR activating mutation or an EGFR gene amplification. In some embodiments, the subject has a c-met activating mutation or a c-met gene amplification. In some embodiments, the cancer is resistant to treatment with one or more of the following ErbB antagonists: an EGFR antagonist, a HER2 antagonist, a HER3 antagonist, or a HER4 antagonist. In some embodiments, the cancer is resistant to treatment with one or more of the following ErbB antagonists: a small molecule therapeutic, a nucleic acid therapeutic, or a protein therapeutic. In some embodiments, the cancer is resistant to treatment with an anti-ErbB antibody. In some embodiments, the cancer is resistant to treatment with an siRNA targeted to an ErbB gene. In some embodiments, the cancer is resistant to treatment with an ErbB kinase inhibitor. In some embodiments, the cancer is resistant to treatment with an EGFR kinase inhibitor. In some embodiments, the cancer is resistant to treatment with one or more of the following EGFR antagonists: gefitinib, erlotinib, lapatinib, PF00299804, CI-1033, EKB-569, BIBW2992, ZD6474, AV-412, EXEL-7647, HKI-272, cetuximab, pantinumumab, or trastuzumab. In some embodiments, one or more of the following ErbB antagonists is administered to the subject: an EGFR antagonist, a HER2 antagonist a HER3 antagonist, or a HER4 antagonist. In some embodiments, one or more of the following ErbB antagonists is administered to the subject: a small molecule therapeutic, a nucleic acid therapeutic, or a protein therapeutic. In some embodiments, one or more of the following EGFR antagonist is administered to the subject: gefitinib, erlotinib, lapatinib, PF00299804, CI-1033, EKB-569, BIBW2992, ZD6474, EXEL-7647, AV-412, HKI-272, cetuximab, pantinumumab, or trastuzumab. In some embodiments, one or more of the following c-met antagonists is administered to the subject: a small molecule therapeutic, a nucleic acid therapeutic, or a protein therapeutic. In some embodiments, one or more of the following c-met antagonists is administered to the subject: PHA-665,752, SU11274, SU5416, PF-02341066, XL-880, MGCD265, XL184, ARQ 197, MP-470, SGX-523, JNJ38877605, AMG 102, or MetMAb. In some embodiments, the ErbB antagonist and the c-met antagonist are administered simultaneously to the subject. In some embodiments, the ErbB antagonist and the c-met antagonist are administered to the subject as a coformulation. In some embodiments, the methods of the invention further comprise administering at least one additional treatment to said subject. In some embodiments, the additional treatment is one or more of the following: administration of an additional therapeutic agent, radiation, photodynamic therapy, laser therapy, or surgery. In some embodiments, the subject is a mammal. In some embodiments, the mammal is a human.

In one aspect, the invention provides methods for treating a subject suffering from a cancer associated with an ErbB activating mutation or an ErbB gene amplification, wherein the subject has developed a resistance to treatment with an ErbB antagonist, comprising determining whether the subject has a c-met activating mutation or a c-met gene amplification, and administering to those subjects having a c-met activating mutation or a c-met gene amplification an ErbB antagonist and a c-met antagonist.

In one aspect, the invention provides methods for treating a subject suffering from a cancer associated with an ErbB activating mutation or an ErbB gene amplification, comprising: (i) monitoring a subject being treated with an ErbB antagonist to determine if the subject develops a c-met activating mutation or a c-met gene amplification, and (ii) modifying the treatment regimen of the subject to include a c-met antagonist in addition to the ErbB antagonist where the subject has developed a c-met activating mutation or a c-met gene amplification.

In one aspect, the invention provides methods for treating a subject suffering from a cancer associated with an ErbB activating mutation or an ErbB gene amplification, comprising: (i) monitoring a subject being treated with ErbB antagonist to determine if the subject develops a resistance to the inhibitor, (ii) testing the subject to determine whether the subject has a c-met activating mutation or a c-met gene amplification, and (iii) modifying the treatment regimen of the subject to include a c-met antagonist in addition to the ErbB antagonist where the subject has a c-met activating mutation or a c-met gene amplification.

In one aspect, the invention provides methods for evaluating an ErbB antagonist, comprising: (i) monitoring a population of subjects being treated with an ErbB antagonist to identify those subjects that develop a resistance to the therapeutic, (ii) testing the resistant subjects to determine whether the subjects have a c-met activating mutation or a c-met gene amplification, and (iii) modifying the treatment regimen of the subjects to include a c-met antagonist in addition to the ErbB antagonist where the subjects have a c-met activating mutation or a c-met gene amplification.

In one aspect, the invention provides methods for reducing ErbB phosphorylation in a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing PI3K mediated signaling in a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing ErbB-mediated signaling in a cancer cell, wherein said cancer cell has acquired resistance to an ErbB antagonist, and wherein said cell comprises a c-met activating mutation or a c-met gene amplification, comprising contacting the cell with a c-met antagonist and an ErbB antagonist.

In one aspect, the invention provides methods for restoring sensitivity of a cancer cell to an ErbB antagonist, wherein said cancer cell has acquired resistance to an ErbB antagonist, and wherein said cell comprises a c-met activating mutation or a c-met gene amplification, comprising contacting the cell with a c-met antagonist and an ErbB antagonist.

In one aspect, the invention provides methods for reducing growth or proliferation of a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for increasing apoptosis of a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing resistance of a cancer cell to an ErbB antagonist, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for treating acquired ErbB antagonist resistance in a cancer cell, wherein said cell comprises a c-met activating mutation or a c-met gene amplification, comprising contacting the cell with a c-met antagonist and an ErbB antagonist.

In some embodiments, the cancer cell is a mammalian cancer cell. In some embodiments, the mammalian cancer cell is a human cancer cell. In some embodiments, the cancer cell is a cell line. In some embodiments, the cancer cell is from a primary tissue sample. In some embodiments, the cancer cell is selected from the group consisting of: a lung cancer cell, a brain cancer cell, a breast cancer cell, a head and neck cancer cell, a colon cancer cell, an ovarian cancer cell, a gastric cancer cell or a pancreatic cancer cell. In some embodiments, the cancer cell is any ErbB-driven cancer. In some embodiments, the cancer cell comprises an ErbB activating mutation. In some embodiments, the ErbB activating mutation is an EGFR activating mutation. In some embodiments, the cancer cell comprises an ErbB gene amplification. In some embodiments, the ErbB gene amplification is an EGFR gene amplification. In some embodiments, the ErbB gene amplification is at least 2-fold. In some embodiments, the c-met amplification is at least 2-fold. In some embodiments, the cancer cell comprises an ErbB gene mutation associated with increased resistance to an ErbB antagonist. In some embodiments, the ErbB gene mutation associated with increased resistance to an ErbB antagonist is a T790M mutation of EGFR. In some embodiments, the ErbB antagonist is selected from the group consisting of: an EGFR antagonist, an HER2 antagonist an HER3 antagonist, or an HER4 antagonist. In some embodiments, the ErbB antagonist is a small molecule therapeutic, a nucleic acid therapeutic, or a protein therapeutic. In some embodiments, the ErbB antagonist is an antibody, an antisense molecule, or a small molecule kinase inhibitor. In some embodiments, the ErbB antagonist is an EGFR kinase inhibitor selected from the group consisting of: gefitinib, erlotinib, lapatinib, PF00299804, CI-1033, EKB-569, BIBW2992, ZD6474, AV-412, HKI-272, EXEL-7647, cetuximab, pantinumumab, or trastuzumab. In some embodiments, the antibody is an anti-EGFR antibody selected from the group consisting of: cetuximab, panitumumab, and trastuzumab. In some embodiments, the nucleic acid therapeutic is an siRNA molecule. In some embodiments, the c-met antagonist is a small molecule therapeutic, a nucleic acid therapeutic, or a protein therapeutic. In some embodiments, the c-met antagonist is an antibody directed against c-met or antibody directed against hepatocyte growth factor (HGF). In some embodiments, the c-met antagonist is PHA-665,752, SU1 1274, SU5416, PF-02341066, XL-880, MGCD265, XL1 84, ARQ 197, MP-470, SGX-523, JNJ38877605, AMG 102, or MetMab. In some embodiments, the nucleic acid therapeutic is an siRNA molecule. In some embodiments, the step of contacting said cell with a c-met antagonist and an ErbB therapeutic is part of a therapeutic regimen that comprises at least one additional treatment modality. In some embodiments, the at least one additional treatment modality is selected from the group consisting of: contacting said cell with one or more additional therapeutic agents, radiation, photodynamic therapy, laser therapy, and surgery.

In one aspect, the invention provides methods for identifying a subject as a candidate for treatment with an ErbB antagonist and a c-met antagonist, wherein said subject has been treated with an ErbB antagonist and suffers from cancer that has acquired resistance to said ErbB antagonist, comprising detecting a c-met activating mutation or c-met gene amplification in a cancer cell from said subject.

In one aspect, the invention provides methods for identifying a c-met antagonist comprising contacting a cancer cell that has acquired resistance to an ErbB antagonist, wherein said cancer cell comprises a c-met activating mutation or a c-met gene amplification, with an ErbB antagonist and a test compound and detecting a change in a cellular process selected from the group consisting of: decreased ErbB phosphorylation, decreased c-met phosphorylation, decreased ErbB-c-met association, decreased EGFR phosphorylation, decreased AKT phosphorylation, decreased cell growth, decreased cell proliferation and increased apoptosis, compared to said cellular process in an identical cell contacted only with an ErbB antagonist.

In one aspect, the invention provides methods for identifying a subject who is being treated with an ErbB antagonist and who is at risk for acquiring resistance to said ErbB antagonist, comprising detecting the presence of a c-met activating mutation or a c-met gene amplification in a cancer cell from said subject, wherein the presence of said c-met activating mutation or c-met gene amplification indicates a risk for acquiring said resistance.

In one aspect, the invention provides methods for producing a cell with acquired resistance to an ErbB antagonist comprising contacting a cell that is sensitive to an ErbB antagonist with at least one ErbB antagonist for at least 4 weeks and identifying cells that acquire resistance to said ErbB antagonist. In some embodiments, the cell does not comprise a mutation in an ErbB gene that confers resistance to said ErbB antagonist.

In one aspect, the invention provides cells produced by a method comprising a method for producing a cell with acquired resistance to an ErbB antagonist comprising contacting a cell that is sensitive to an ErbB antagonist with at least one ErbB antagonist for at least 4 weeks and identifying cells that acquire resistance to said ErbB antagonist.

In one aspect, the invention provides methods for treating a subject suffering from a cancer that is resistant to treatment with an ErbB antagonist, comprising administering to the subject an ErbB antagonist and an agent that inhibits HGF mediated activation of c-met.

In some embodiments, the agent is an antibody that prevents HGF from binding to c-met. In some embodiments, the antibody is an anti-HGF antibody. In some embodiments, the antibody is an anti-c-met antibody. In some embodiments, the ErbB is HER3. In some embodiments, the ErbB antagonist is an HER3 antagonist. In some embodiments, the cancer cell's growth and/or survival is promoted by ErbB.

In one aspect, the invention provides methods for treating a subject suffering from a cancer associated with an ErbB activating mutation or an ErbB gene amplification, wherein the subject has developed a resistance to treatment with an ErbB antagonist, comprising determining whether the subject has elevated c-met levels and/or activity, and administering to those subjects having elevated c-met activity an ErbB antagonist and a c-met antagonist.

In one aspect, the invention provides methods for treating a subject suffering from a cancer associated with an ErbB activating mutation or an ErbB gene amplification, comprising: (i) monitoring a subject being treated with an ErbB antagonist to determine if the subject develops elevated levels and/or c-met activity, and (ii) modifying the treatment regimen of the subject to include a c-met antagonist in addition to the ErbB antagonist where the subject has developed elevated c-met levels and/or activity.

In one aspect, the invention provides methods for treating a subject suffering from a cancer associated with an ErbB activating mutation or an ErbB gene amplification, comprising: (i) monitoring a subject being treated with ErbB antagonist to determine if the subject develops a resistance to the inhibitor, (ii) testing the subject to determine whether the subject has elevated c-met levels and/or activity, and (iii) modifying the treatment regimen of the subject to include a c-met antagonist in addition to the ErbB antagonist where the subject has elevated c-met levels and/or activity.

In some embodiments, the elevated c-met activity is associated with a c-met gene amplification, a c-met activating mutation, or HGF mediated c-met activation. In some embodiments, the HGF mediated c-met activation is associated with elevated HGF expression levels or elevated HGF activity. In some embodiments, the HGF mediated c-met activation is associated with an HGF gene amplification or an HGF activating mutation. In some embodiments, the c-met antagonist is an agent that inhibits HGF mediated activation of c-met. In some embodiments, the agent is an antibody that prevents HGF from binding to c-met. In some embodiments, the antibody is an anti-HGF antibody or an anti-c-met antibody.

In another aspect, the invention provides a method for reducing ErbB phosphorylation in a cancer cell by contacting the cell with an ErbB antagonist and a c-met antagonist.

In certain embodiments, the cancer cell has acquired a resistance to an ErbB antagonist and comprises elevated levels of c-met activity and/or expression, e.g., associated with, for example, an activating mutation in the c-met gene, a c-met gene amplification, or HGF mediated c-met activation. The methods disclosed herein may be used to reduce the phosphorylation of one or more of EGFR, HER2, HER3 and/or HER4.

In certain embodiments, it may be desirable to compare the level of ErbB phosphorylation in the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay, etc.

In another aspect, the invention provides a method for reducing PI3K mediated signaling in a cancer cell by contacting the cell with an ErbB antagonist and a c-met antagonist. In exemplary embodiments, the cancer cell has acquired a resistance to an ErbB antagonist and comprises elevated levels of c-met activity and/or expression (e.g., associated with an activating mutation in the c-met gene, a c-met gene amplification, or HGF mediated c-met activation). In certain embodiments, it may be desirable to compare the level of PDK mediated signaling in the cancer cell to a control, e.g., a cell that has not been contacted with an ErbB antagonist, a c-met antagonist or both, or a cell that has been contacted with a different amount of one or both of the therapeutic agents, or a reference value, such as an expected value for a given assay, etc.

In another aspect, the invention provides a method for reducing ErbB-mediated signaling in a cancer cell by contacting the cell with an ErbB antagonist and a c-met antagonist. In exemplary embodiments, the cancer cell has acquired a resistance to an ErbB antagonist and comprises elevated levels of c-met activity and/or expression, for example, associated with an activating mutation in the c-met gene, a c-met gene amplification, or HGF mediated c-met activation. The methods disclosed herein may be used to reduce signaling mediated by one or more of EGFR, HER2, HER3 and/or HER4. In certain embodiments, it may be desirable to compare the level of ErbB-mediated signaling in the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay, etc.

In another aspect, the invention provides a method for (i) restoring the sensitivity of a cancer cell to an ErbB antagonist, (ii) reducing resistance of a cancer cell to an ErbB antagonist, and/or (iii) treating acquired ErbB antagonist resistance in a cancer cell, by contacting the cell with an ErbB antagonist and a c-met antagonist. In exemplary embodiments, the cancer cell has acquired a resistance to an ErbB antagonist and comprises elevated levels of c-met activity and/or expression, e.g., associated with an activating mutation in the c-met gene, a c-met gene amplification, or HGF mediated c-met activation. The methods disclosed herein may be used to restore the sensitivity, reduce the resistance, and/or treat an acquired resistance, of a cancer cell to one or more of the following: an EGFR antagonist, an HER2 antagonist, an HER3 antagonist and/or an HER4 antagonist.

For example, an amount of cell growth and/or proliferation and/or amount of apoptosis may be determined in the presence of the ErbB antagonist/ c-met antagonist combination therapy as compared to the ErbB antagonist alone. A decrease in the cell growth and/or proliferation and/or an increase in apoptosis of the cancer cell is indicative of an increase in sensitivity, or a reduction in resistance, to the ErbB antagonist.

In another aspect, the invention provides a method for reducing growth and/or proliferation of a cancer cell, or increasing apoptosis of a cancer cell, by contacting the cell with an ErbB antagonist and a c-met antagonist. In exemplary embodiments, the cancer cell has acquired a resistance to an ErbB antagonist and comprises elevated c-met activity and/or expression, e.g., associated with an activating mutation in the c-met gene, a c-met gene amplification, or HGF mediated c-met activation. In certain embodiments, it may be desirable to compare the level of growth and/or proliferation and/or apoptosis of the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay.

The c-met antagonist can be administered serially or in combination with the HER antagonist, either in the same composition or as separate compositions. The administration of the c-met antagonist and the HER antagonist can be done simultaneously, e.g., as a single composition or as two or more distinct compositions, using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the HER antagonist may be administered first, followed by the c-met antagonist. However, simultaneous administration or administration of the c-met antagonist first is also contemplated. Accordingly, in one aspect, the invention provides methods comprising administration of a c-met antagonist (such as an anti-c-met antibody), followed by administration of an HER antagonist. In certain embodiments, intervals ranging from minutes to days, to weeks to months, can be present between the administrations of the two or more compositions.

In one aspect, the invention provides a composition for use in treating a cancer comprising an effective amount of a c-met antagonist and a pharmaceutically acceptable carrier, wherein said use comprises simultaneous or sequential administration of an HER antagonist. In some embodiments, the c-met antagonist is an anti-c-met antibody.

In one aspect, the invention provides a composition for use in treating a cancer comprising an effective amount of a c-met antagonist and a pharmaceutically acceptable carrier, wherein said use comprises simultaneous or sequential administration of an HER antagonist. In some embodiments, the c-met antagonist is an anti-c-met antibody.

Depending on the specific cancer indication to be treated, the combination therapy of the invention can be combined with additional therapeutic agents, such as chemotherapeutic agents, or additional therapies such as radiotherapy or surgery. Many known chemotherapeutic agents can be used in the combination therapy of the invention. Preferably those chemotherapeutic agents that are standard for the treatment of the specific indications will be used. Dosage or frequency of each therapeutic agent to be used in the combination is preferably the same as, or less than, the dosage or frequency of the corresponding agent when used without the other agent(s).

### BRIEF DESCRIPTION OF THE DRAWINGS

FIG. 1: provides a schematic of the HER2 protein structure, and amino acid sequences for Domains I-IV (SEQ ID Nos.19-22, respectively) of the extracellular domain thereof.

FIGS. 2A and 2B: depict alignments of the amino acid sequences of the variable light (VL) (FIG. 2A) and variable heavy (VH) (FIG. 2B) domains of murine monoclonal antibody 2C4 (SEQ ID Nos. 1 and 2, respectively); VL and VH domains of humanized 2C4 version 574 (SEQ ID Nos. 3 and 4, respectively), and human VL and VH consensus frameworks (hum κ1, light kappa subgroup I; humIII, heavy subgroup III) (SEQ ID Nos. 5 and 6, respectively). Asterisks identify differences between humanized 2C4 version 574 and murine monoclonal antibody 2C4 or between humanized 2C4 version 574 and the human framework. Complementarity Determining Regions (CDRs) are in brackets.

FIGS. 3A and 3B: show the amino acid sequences of Pertuzumab light chain (SEQ ID No. 15) and heavy chain (SEQ ID No. 16). CDRs are shown in bold. The carbohydrate moiety is attached to Asn 299 of the heavy chain.

FIGS. 4A and 4B: show the amino acid sequences of Pertuzumab light chain (SEQ ID No. 17) and heavy chain (SEQ ID No. 18), each including an intact amino terminal signal peptide sequence.

FIG. 5: depicts, schematically, binding of 2C4 at the heterodimeric binding site of HER2, thereby preventing heterodimerization with activated EGFR or HER3.

FIG. 6: depicts coupling of HER2/HER3 to the MAPK and Akt pathways.

FIG. 7: compares activities of Trastuzumab and Pertuzumab.

FIGS. 8A and 8B: show the amino acid sequences of Trastuzumab light chain (SEQ ID No. 13) and heavy chain (SEQ ID No. 14).

FIGS. 9A and 9B: depict a variant Pertuzumab light chain sequence (SEQ ID No. 51) and a variant Pertuzumab heavy chain sequence (SEQ ID No. 52).

FIG. 10: EBC1 shMet 4.12 cells (shMet 4.12) containing a tetracycline inducible shRNA directed against met or control shRNA directed against GFP (shGFP2) were grown in control media (Con) or media with 0.1 ug/ml tetracycline analog Doxicycline (Dox) for 48 hours. After serum-starvation for 2 hours, cells were untreated (−) or treated with TGFα (T, 20 nM) or Heregulin b1 (Hrg, 2 nM) for 20 minutes. Whole cell lysates were evaluated for expression of total and phospho-proteins as indicated.

FIG. 11: NSCLC H441 cells containing an inducible shRNA directed against c-met or control shRNA directed against GFP were grown in control media or media containing 0.1 ug/ml Dox (Dox) for 48 hours. After serum-starvation for 2 hours, cells were untreated (−) or treated with TGFα (T) or Heregulin b1 (H) for 20 minutes.

FIG. 12: depicts amino acid sequences of the framework regions (FR), hypervariable regions (HVR), first constant domain (CL or CH1) and Fc region (Fc) of one embodiment of an anti-c-met antibody. The Fc sequence depicted comprises mutations T366S, L368A and Y407V, as described in WO 2005/063816.

FIG. 13: depicts sequence of an Fc polypeptide comprising mutation T366W, as described in WO 2005/063816. In one embodiment, an Fc polypeptide comprising this sequence forms a complex with an Fc polypeptide comprising the Fc sequence of FIG. 7 to generate an Fc region.

FIGS. 14A-C: (A) EBCshMet 4.12 or EBCshGFP2 cells were untreated (−) or treated with Dox (+) for 24, 48 or 72 hours. Protein lysates were evaluated for Met, pEGFR or Her3 by western blotting. (B) EBCshMet 4.12 cells were treated with Dox (100 ng/ml) for 48 hours and analyzed by FACS for cell surface Her3. (C) Mice with EBCshMet 4.12 tumors were given drinking water with 1 mg/ml Dox in 5% sucrose (Dox) or 5% sucrose alone (Sucrose) for 3 days. Tumors lysates were evaluated for Her3 protein by western blotting.

FIG. 15: Combination efficacy of pertuzumab with shRNA knockdown of c-Met in the EBC-1 NSCLC xenograft model. EBC-1-shMet-4.5 tumors were established in nude (CRL nu/nu) animals and then treated with either drinking water containing 5% sucrose, 1 mg/mL doxycycline (Dox) in the drinking water formulated in 5% sucrose, pertuzumab, or pertuzumab plus 1 mg/mL doxycycline in the drinking water formulated in 5% sucrose. Sucrose or Dox water was maintained throughout the study with bottles being interchanged every 2-3 days. Tumor volumes and SEM were calculated as described in the Examples.

### DETAILED DESCRIPTION

### I. Definitions

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur. The term “wild type HGF” generally refers to a polypeptide comprising the amino acid sequence of a naturally occurring HGF protein. The term “wild type HGF sequence” generally refers to an amino acid sequence found in a naturally occurring HGF. C-met is a known receptor for HGF through which HGF intracellular signaling is biologically effectuated.

The term “HGF variant” as used herein refers to a HGF polypeptide which includes one or more amino acid mutations in the native HGF sequence. Optionally, the one or more amino acid mutations include amino acid substitution(s).

A “native sequence” polypeptide comprises a polypeptide having the same amino acid sequence as a polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

By “EGFR” (interchangeably termed “ErbB1”, “HER1” and “epidermal growth factor receptor”) is meant the receptor tyrosine kinase polypeptide Epidermal Growth Factor Receptor which is described in Ullrich et al, Nature (1984) 309:418425, alternatively referred to as Her-1 and the c-erbB gene product, as well as variants thereof such as EGFRvIII. Variants of EGFR also include deletional, substitutional and insertional variants, for example those described in Lynch et al (New England Journal of Medicine 2004, 350:2129), Paez et al (Science 2004, 304:1497), Pao et al (PNAS 2004, 101:13306).

A “HER receptor” is a receptor protein tyrosine kinase which belongs to the HER receptor family and includes HER2 and HER3 receptors but specifically excludes EGFR and HER4 receptor. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an amino acid sequence “variant” thereof. Preferably the HER receptor is native sequence human HER receptor.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

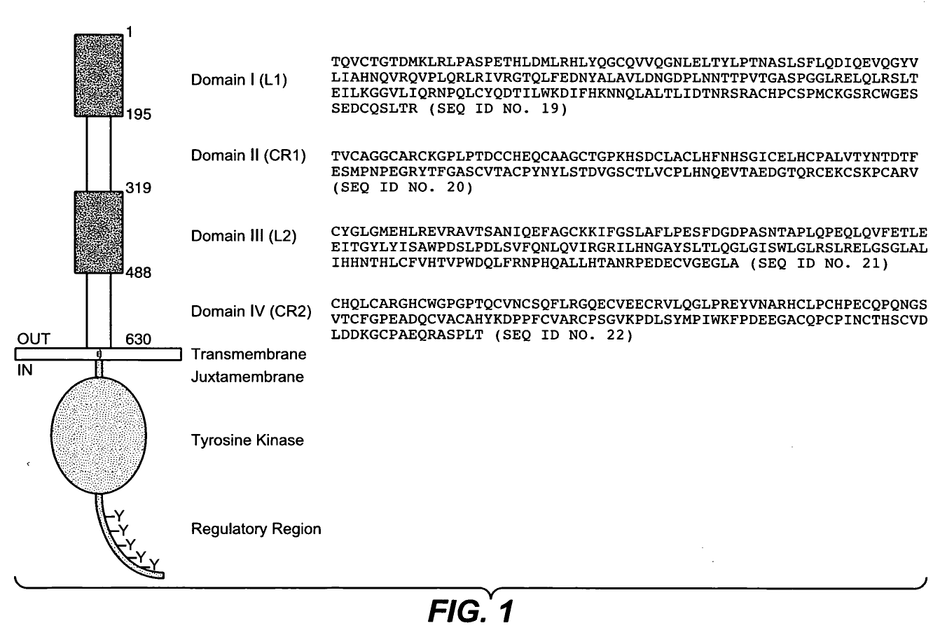

Herein, “HER2 extracellular domain” or “HER2 ECD” refers to a domain of HER2 that is outside of a cell, either anchored to a cell membrane, or in circulation, including fragments thereof. In one embodiment, the extracellular domain of HER2 may comprise four domains: “Domain I” (amino acid residues from about 1-195; SEQ ID NO:19), “Domain II” (amino acid residues from about 196-319; SEQ ID NO:20), “Domain III” (amino acid residues from about 320-488: SEQ ID NO:21), and “Domain IV” (amino acid residues from about 489-630; SEQ ID NO:22) (residue numbering without signal peptide). See Garrett et al. Mol Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 1 herein.

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989).

The terms “ErbB4” and “HER4” herein refer to the receptor polypeptide as disclosed, for example, in EP Pat Appln No 599,274; Plowman et al., Proc. Natl. Acad. Sci. USA, 90:1746-1750 (1993); and Plowman et al., Nature, 366:473-475 (1993), including isoforms thereof, e.g., as disclosed in WO99/19488, published Apr. 22, 1999.

As used herein, “ErbB” refers to the receptor polypeptides EGFR, HER2, HER3, and HER4.

A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Other proteins, such as a cytokine receptor subunit (e.g. gp130) may be associated with the dimer. Preferably, the HER dimer comprises HER2.

A “HER heterodimer” herein is a noncovalently associated heterodimer comprising at least two different HER receptors selected from EGFR, HER2 and HER3. Exemplary HER heterodimers include EGFR-HER2, or HER2-HER3 heterodimers.

By “HER ligand” is meant a polypeptide which binds to and/or activates a HER2 or HER3 receptor. The HER ligand of particular interest herein is a native sequence human HER ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanoma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Commun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)); a heregulin (see below); neuregulin-2 (NRG-2) (Carraway et al., Nature 387:512-516 (1997)); neuregulin-3 (NRG-3) (Zhang et al., Proc. Natl. Acad. Sci. 94:9562-9567 (1997)); neuregulin-4 (NRG-4) (Harari et al. Oncogene 18:2681-89 (1999)) or cripto (CR-1) (Kannan et al. J. Biol. Chem. 272(6):3330-3335 (1997) HER ligands which bind EGFR include EGF, TGF-α, amphiregulin, betacellulin, HB-EGF and epiregulin. HER ligands which bind HER3 include heregulins.

“Heregulin” (HRG) when used herein refers to a polypeptide encoded by the heregulin gene product as disclosed in U.S. Pat. No. 5,641,869, or Marchionni et al., Nature, 362:312-318 (1993). Examples of heregulins include heregulin-α, heregulin-β1, heregulin-β2 and heregulin-β3 (Holmes et al., Science, 256:1205-1210 (1992); and U.S. Pat. No. 5,641,869); neu differentiation factor (NDF) (Peles et al. Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); γ-heregulin (Schaefer et al. Oncogene 15:1385-1394 (1997)).

A “HER antagonist” (interchangeably termed “HER inhibitor”) is an agent which interferes with HER activation or function. Examples of HER inhibitors include HER antibodies (e.g. HER2, HER3 antibodies); small molecule HER antagonists; HER tyrosine kinase inhibitors. Preferably, the HER inhibitor is an antibody or small molecule which binds to a HER receptor. In a particular embodiment, a HER inhibitor has a binding affinity (dissociation constant) to HER of about 1,000 nM or less. In another embodiment, a HER inhibitor has a binding affinity to HER of about 100 nM or less. In another embodiment, a HER inhibitor has a binding affinity to HER of about 50 nM or less. In a particular embodiment, a HER inhibitor is covalently bound to HER In a particular embodiment, a HER inhibitor inhibits HER signaling with an IC50 of 1,000 nM or less. In another embodiment, a HER inhibitor inhibits HER signaling with an IC50 of 500 nM or less. In another embodiment, a HER inhibitor inhibits HER signaling with an IC50 of 50 nM or less.

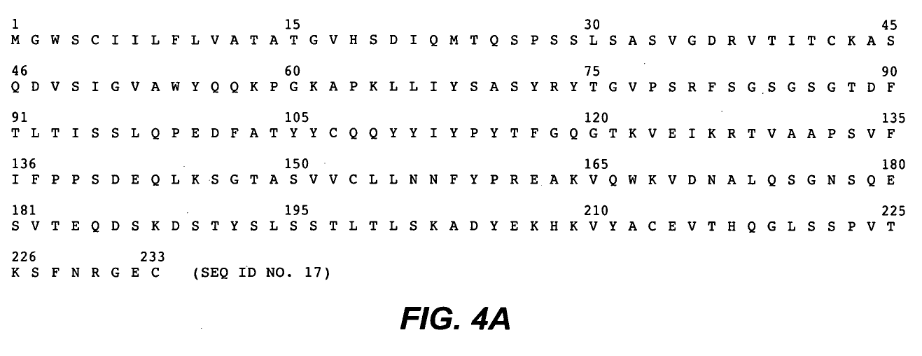

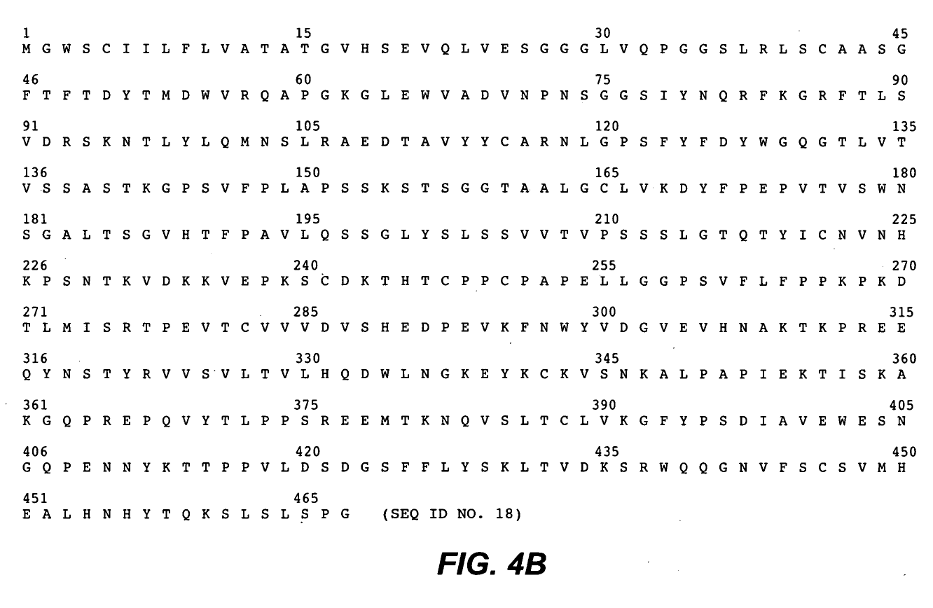

A “HER dimerization inhibitor” is an agent which inhibits formation of a HER dimer or HER heterodimer. Preferably, the HER dimerization inhibitor is an antibody, for example an antibody which binds to HER2 at the heterodimeric binding site thereof. The most preferred HER dimerization inhibitor herein is pertuzumab or MAb 2C4. Binding of 2C4 to the heterodimeric binding site of HER2 is illustrated in FIG. 4. Other examples of HER dimerization inhibitors include antibodies which bind to HER3 and inhibit dimerization thereof with one or more other HER receptors; antisense dimerization inhibitors; etc.

A “HER2 dimerization inhibitor” is an agent that inhibits formation of a dimer or heterodimer comprising HER2.

“HER activation” refers to activation, or phosphorylation, of any one or more HER 2 or HER3 receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“c-met activation” refers to activation, or phosphorylation, of c-met. Generally, c-met activation results in signal transduction (e.g. that caused by an intracellular kinase domain of c-met receptor phosphorylating tyrosine residues in c-met or a substrate polypeptide). C-met activation may be mediated by c-met ligand (e.g., HGF) binding to a c-met dimer. C-met ligand binding to a c-met dimer may activate a kinase domain of one or more of the c-met in the dimer and thereby results in phosphorylation of tyrosine residues in one or more of the c-met and/or phosphorylation of tyrosine residues in additional substrate polypeptides(s).

“Phosphorylation” refers to the addition of one or more phosphate group(s) to a protein, such as a HER receptor, or substrate thereof.

An antibody which “inhibits HER2 and/or HER3 dimerization” is an antibody which inhibits, or interferes with, formation of a HER2 and/or HER3 dimer. Preferably, such an antibody binds to HER2 at the heterodimeric binding site thereof The most preferred dimerization inhibiting antibody herein is pertuzumab or MAb 2C4.

A HER2 antibody that “binds to a heterodimeric binding site” of HER2, binds to residues in domain II (and optionally also binds to residues in other of the domains of the HER2 extracellular domain, such as domains I and III), and can sterically hinder, at least to some extent, formation of a HER2-EGFR, or a HER2-HER3 heterodimer. Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II and optionally residues in other domain(s) of HER2, such as domains I and III. Preferably the antibody that binds to domain II binds to the junction between domains I, II and III of HER2.

An antibody which “inhibits HER dimerization” is an antibody which inhibits, or interferes with, formation of a HER dimer. Preferably, such an antibody binds to HER2 at the heterodimeric binding site thereof. The most preferred dimerization inhibiting antibody herein is pertuzumab or MAb 2C4. Binding of 2C4 to the heterodimeric binding site of HER2 is illustrated in FIG. 4. Other examples of antibodies which inhibit HER dimerization are antibodies which bind to HER3 and inhibit dimerization thereof with one or more other HER receptors.

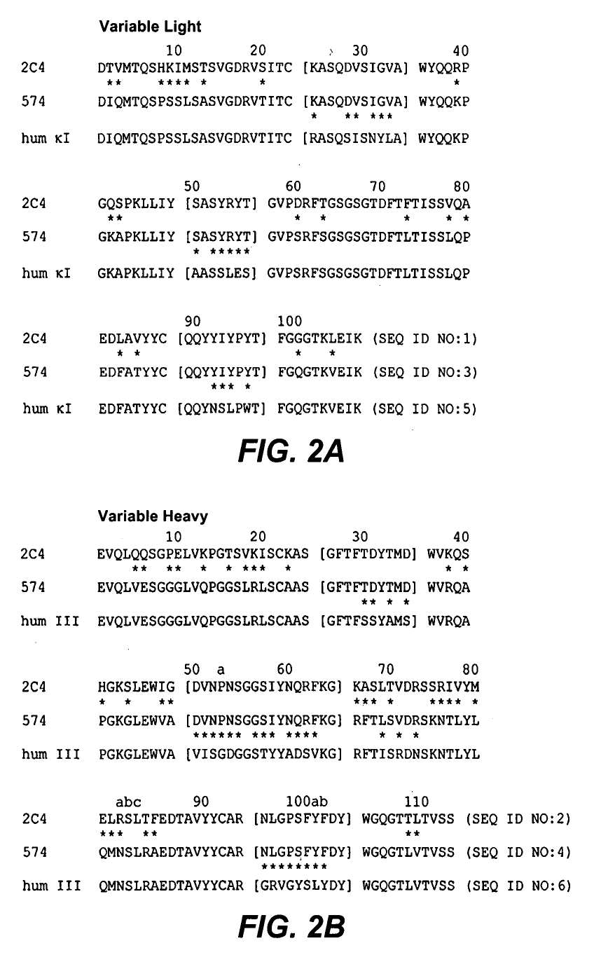

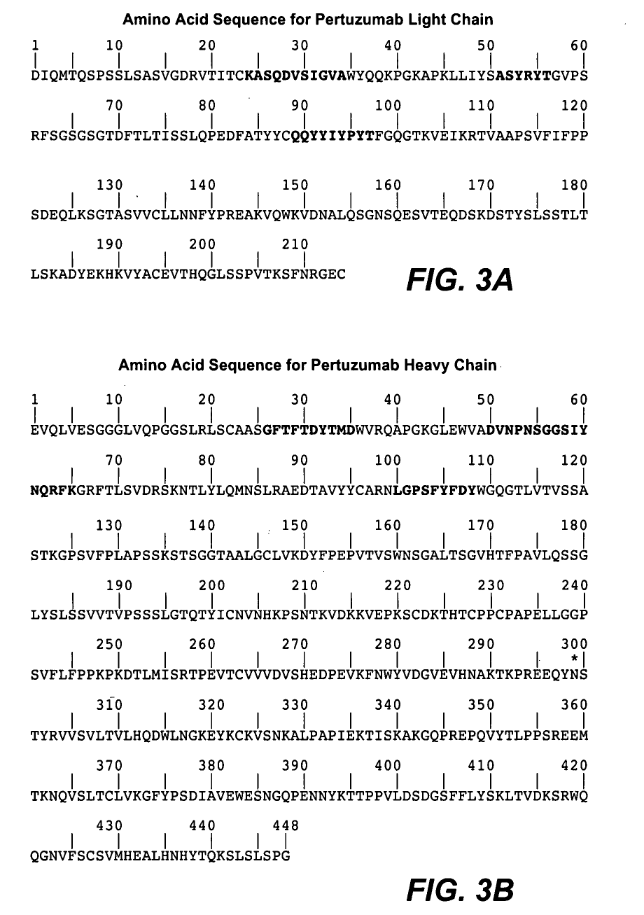

An antibody which “blocks ligand activation of a HER receptor more effectively than trastuzumab” is one which reduces or eliminates HER ligand activation of HER receptor(s) or HER dimer(s) more effectively (for example at least about 2-fold more effectively) than trastuzumab. Preferably, such an antibody blocks HER ligand activation of a HER receptor at least about as effectively as murine monoclonal antibody 2C4 or a Fab fragment thereof, or as pertuzumab or a Fab fragment thereof. One can evaluate the ability of an antibody to block ligand activation of a HER receptor by studying HER dimers directly, or by evaluating HER activation, or downstream signaling, which results from HER dimerization, and/or by evaluating the antibody-HER2 binding site, etc. Assays for screening for antibodies with the ability to inhibit ligand activation of a HER receptor more effectively than trastuzumab are described in Agus et al. Cancer Cell 2: 127-137 (2002) and U.S. Pat. No. 6,949,245 (Adams et al). By way of example only, one may assay for: inhibition of HER dimer formation (see, e.g., FIGS. 1A-B of Agus et al. Cancer Cell 2: 127-137 (2002); and U.S. Pat. No. 6,949,245); reduction in HER ligand activation of cells which express HER dimers (U.S. Pat. No. 6,949,245 and FIGS. 2A-B of Agus et al. Cancer Cell 2: 127-137 (2002), for example); blocking of HER ligand binding to cells which express HER dimers (U.S. Pat. No. 6,949,245, and FIG. 2E of Agus et al. Cancer Cell 2: 127-137 (2002), for example); cell growth inhibition of cancer cells (e.g. MCF7, MDA-MD-134, ZR-75-1, MD-MB-175, T-47D cells) which express HER dimers in the presence (or absence) of HER ligand (U.S. Pat. No. 6,949,245and FIGS. 3A-D of Agus et al. Cancer Cell 2: 127-137 (2002), for instance); inhibition of downstream signaling (for instance, inhibition of HRG-dependent AKT phosphorylation or inhibition of HRG- or TGFα-dependent MAPK phosphorylation) (see, U.S. Pat. No. 6,949,245, and FIGS. 2C-D of Agus et al. Cancer Cell 2: 127-137 (2002), for example). One may also assess whether the antibody inhibits HER dimerization by studying the antibody-HER2 binding site, for instance, by evaluating a structure or model, such as a crystal structure, of the antibody bound to HER2 (See, for example, Franklin et al. Cancer Cell 5:317-328 (2004)).

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2. Franklin et al. Cancer Cell 5:317-328 (2004).

The HER2 antibody may “inhibit HRG-dependent AKT phosphorylation” and/or inhibit “HRG- or TGFα-dependent MAPK phosphorylation” more effectively (for instance at least 2-fold more effectively) than trastuzumab (see Agus et al. Cancer Cell 2: 127-137 (2002) and U.S. Pat. No. 6,949,245, by way of example).

As used herein, the term “EGFR-targeted drug” refers to a therapeutic agent that binds to EGFR and inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small molecules that bind to EGFR include ZD1839 or Gefitinib (IRESSA; Astra Zeneca); CP-358774 or Erlotinib (TARCEVA™; Genentech/OSI); and AG1478, AG1571 (SU 5271; Sugen); EMD-7200.

As used herein, the term “c-met-targeted drug” refers to a therapeutic agent that binds to c-met and inhibits c-met activation. An example of a c-met targeted drug is MetMAb (OA5D5.v2).

As used herein, the term “HER-targeted drug” refers to a therapeutic agent that binds to HER and inhibits HER activation.

By “EGFR resistant” cancer is meant that the cancer patient has progressed while receiving an EGFR antagonist therapy (i.e., the patient is “EGFR refractory”), or the patient has progressed within 12 months (for instance, within one, two, three, or six months) after completing an EGFR antagonist-based therapy regimen. For example, cancers which incorporate T790M mutant EGFR are resistant to erlotinib and gefitinib therapy.

By “erlotinib or gefitinib resistant” cancer is meant that the cancer patient has progressed while receiving erlotinib- or gefitinib-based therapy (i.e., the patient is “erlotinib or gefitinib refractory”), or the patient has progressed within 12 months (for instance, within one, two, three, or six months) after completing an erlotinib- or gefitinib-based therapy regimen.

A “c-met antagonist” (interchangeably termed “c-met inhibitor”) is an agent that interferes with c-met activation or function. Examples of c-met inhibitors include c-met antibodies; HGF antibodies; small molecule c-met antagonists; c-met tyrosine kinase inhibitors; antisense and inhibitory RNA (e.g., shRNA) molecules (see, for example, WO2004/87207). Preferably, the c-met inhibitor is an antibody or small molecule which binds to c-met. In a particular embodiment, a c-met inhibitor has a binding affinity (dissociation constant) to c-met of about 1,000 nM or less. In another embodiment, a c-met inhibitor has a binding affinity to c-met of about 100 nM or less. In another embodiment, a c-met inhibitor has a binding affinity to c-met of about 50 nM or less. In a particular embodiment, a c-met inhibitor is covalently bound to c-met. In a particular embodiment, a c-met inhibitor inhibits c-met signaling with an IC50 of 1,000 nM or less. In another embodiment, a c-met inhibitor inhibits c-met signaling with an IC50 of 500 nM or less. In another embodiment, a c-met inhibitor inhibits c-met signaling with an IC50 of 50 nM or less.

“C-met activation” refers to activation, or phosphorylation, of the c-met receptor. Generally, c-met activation results in signal transduction (e.g. that caused by an intracellular kinase domain of a c-met receptor phosphorylating tyrosine residues in c-met or a substrate polypeptide). C-met activation may be mediated by c-met ligand (HGF) binding to a c-met receptor of interest. HGF binding to c-met may activate a kinase domain of c-met and thereby result in phosphorylation of tyrosine residues in the c-met and/or phosphorylation of tyrosine residues in additional substrate polypeptides(s).

For the purposes herein, “Trastuzumab,” “HERCEPTIN®,” and “huMAb4D5-8” refer to an antibody comprising the light and heavy chain amino acid sequences in SEQ ID NOS. 13 and 14, respectively.

Herein, “Pertuzumab” and “rhuMAb 2C4,” refer to an antibody comprising the variable light and variable heavy amino acid sequences in SEQ ID Nos. 3 and 4, respectfully. Where Pertuzumab is an intact antibody, it preferably comprises the light chain and heavy chain amino acid sequences in SEQ ID Nos. 15 and 16, respectively.

The HER2 antibody may be one which does “not inhibit HER2 ectodomain cleavage” (Molina et al. Cancer Res. 61:4744-4749(2001)).

A HER2 antibody that “binds to a heterodimeric binding site” of HER2, binds to residues in domain II (and optionally also binds to residues in other of the domains of the HER2 extracellular domain, such as domains I and III), and can sterically hinder, at least to some extent, formation of a HER2-EGFR, HER2-HER3, or HER2-HER4 heterodimer. Franklin et al. Cancer Cell 5:317-328 (2004) characterize the HER2-Pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II and optionally residues in other domain(s) of HER2, such as domains I and III.

A “HER positive cancer” is one comprising cells which have HER protein present at their cell surface.

The “epitope 2C4” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2 using methods known in the art and/or one can study the antibody-HER2 structure (Franklin et al. Cancer Cell 5:317-328 (2004)) to see what domain(s) of HER2 is/are bound by the antibody. Epitope 2C4 comprises residues from domain II in the extracellular domain of HER2. 2C4 and Pertuzumab bind to the extracellular domain of HER2 at the junction of domains I, II and III. Franklin et al. Cancer Cell 5:317-328 (2004).

The “epitope 4D5” is the region in the extracellular domain of HER2 to which the antibody 4D5 (ATCC CRL 10463) and Trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive, in FIG. 1).

The “epitope 7C2/7F3” is the region at the N terminus, within Domain I, of the extracellular domain of HER2 to which the 7C2 and/or 7F3 antibodies (each deposited with the ATCC, see below) bind. To screen for antibodies which bind to the 7C2/7F3 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to establish whether the antibody binds to the 7C2/7F3 epitope on HER2 (e.g. any one or more of residues in the region from about residue 22 to about residue 53 of HER2 in FIG. 1).

A “biological sample” (interchangeably termed “sample” or “tissue or cell sample”) encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a primary or metastatic tumor. The biological sample may contain compounds which are not naturally intermixed with the tissue in nature such as preservatives, anticoagulants, buffers, fixatives, nutrients, antibiotics, or the like.

The term “ligand-independent” as used herein, as for example applied to receptor signaling activity, refers to signaling activity that is not dependent on the presence of a ligand. A receptor having ligand-independent kinase activity will not necessarily preclude the binding of ligand to that receptor to produce additional activation of the kinase activity.

The term “constitutive” as used herein, as for example applied to receptor kinase activity, refers to continuous signaling activity of a receptor that is not dependent on the presence of a ligand or other activating molecules. For example, EGFR variant III (EGFRvIII) which is commonly found in glioblastoma multiforme has deleted much of its extracellular domain. Although ligands are unable to bind EGFRvIII it is nevertheless continuously active and is associated with abnormal proliferation and survival. Depending on the nature of the receptor, all of the activity may be constitutive or the activity of the receptor may be further activated by the binding of other molecules (e. g. ligands). Cellular events that lead to activation of receptors are well known among those of ordinary skill in the art. For example, activation may include oligomerization, e.g., dimerization, trimerization, etc., into higher order receptor complexes. Complexes may comprise a single species of protein, i.e., a homomeric complex. Alternatively, complexes may comprise at least two different protein species, i.e., a heteromeric complex. Complex formation may be caused by, for example, overexpression of normal or mutant forms of receptor on the surface of a cell. Complex formation may also be caused by a specific mutation or mutations in a receptor.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

Protein “expression” refers to conversion of the information encoded in a gene into messenger RNA (mRNA) and then to the protein.

Herein, a sample or cell that “expresses” a protein of interest (such as a HER receptor or HER ligand) is one in which mRNA encoding the protein, or the protein, including fragments thereof, is determined to be present in the sample or cell.

A “tyrosine kinase inhibitor” is a molecule which inhibits to some extent tyrosine kinase activity of a tyrosine kinase such as a c-met receptor.

A cancer or biological sample which “displays c-met and/or HER expression, amplification, or activation” is one which, in a diagnostic test, expresses (including overexpresses) c-met and/or HER, has amplified c-met and/or HER gene, and/or otherwise demonstrates activation or phosphorylation of a c-met and/or HER.

A cancer or biological sample which “does not display c-met and/or HER expression, amplification, or activation” is one which, in a diagnostic test, does not express (including overexpress) c-met and/or HER, does not have amplified c-met and/or HER gene, and/or otherwise does not demonstrate activation or phosphorylation of a c-met and/or HER.

A cancer or biological sample which “displays c-met and/or HER activation” is one which, in a diagnostic test, demonstrates activation or phosphorylation of c-met and/or HER. Such activation can be determined directly (e.g. by measuring c-met and/or HER phosphorylation by HER) or indirectly.

A cancer or biological sample which “does not display c-met and/or HER activation” is one which, in a diagnostic test, does not demonstrate activation or phosphorylation of a c-met and/or HER. Such activation can be determined directly (e.g. by measuring c-met and/or HER phosphorylation by ELISA) or indirectly.

A cancer cell with “c-met and/or HER overexpression or amplification” is one which has significantly higher levels of a c-met and/or HER protein or gene compared to a noncancerous cell of the same tissue type. Such overexpression may be caused by gene amplification or by increased transcription or translation. c-met and/or HER overexpression or amplification may be determined in a diagnostic or prognostic assay by evaluating increased levels of the c-met and/or HER protein present on the surface of a cell (e.g. via an immunohistochemistry assay; IHC). Alternatively, or additionally, one may measure levels of c-met and/or HER -encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A cancer cell which “does not overexpress or amplify c-met and/or HER” is one which does not have higher than normal levels of c-met and/or HER protein or gene compared to a noncancerous cell of the same tissue type.

The term “mutation”, as used herein, means a difference in the amino acid or nucleic acid sequence of a particular protein or nucleic acid (gene, RNA) relative to the wild-type protein or nucleic acid, respectively. A mutated protein or nucleic acid can be expressed from or found on one allele (heterozygous) or both alleles (homozygous) of a gene, and may be somatic or germ line. In the instant invention, mutations are generally somatic. Mutations include sequence rearrangements such as insertions, deletions, and point mutations (including single nucleotide/amino acid polymorphisms).

Protein “expression” refers to conversion of the information encoded in a gene into messenger RNA (mRNA) and then to the protein.

Herein, a sample or cell that “expresses” a protein of interest (such as a HER receptor or HER ligand) is one in which mRNA encoding the protein, or the protein, including fragments thereof, is determined to be present in the sample or cell.

To “inhibit” is to decrease or reduce an activity, function, and/or amount as compared to a reference.

An “immunoconjugate” (interchangeably referred to as “antibody-drug conjugate,” or “ADC”) means an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The Fc sequence of an immunoglobulin generally comprises two constant domains, a CH2 domain and a CH3 domain, and optionally comprises a CH4 domain. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

By “Fc polypeptide” herein is meant one of the polypeptides that make up an Fc region. An Fc polypeptide may be obtained from any suitable immunoglobulin, such as IgG₁, IgG₂, IgG₃, or IgG₄ subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. In one embodiment, an antibody of the invention is a one-armed antibody as described in WO2005/063816. In one embodiment, the one-armed antibody comprises Fc mutations constituting “knobs” and “holes” as described in WO2005/063816. For example, a hole mutation can be one or more of T366A, L368A and/or Y407V in an Fc polypeptide, and a cavity mutation can be T366W.

A “blocking” antibody or an antibody “antagonist” is one which inhibits or reduces biological activity of the antigen it binds. Preferred blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRHi of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004) Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al, Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boemer et al., J. Immunol., 147 (1):86-95 (1991); and U.S. Pat. No. 5,750,373.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

To increase the half-life of the antibodies or polypeptide containing the amino acid sequences of this invention, one can attach a salvage receptor binding epitope to the antibody (especially an antibody fragment), as described, e.g., in U.S. Pat. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (e.g., Ghetie et al., Ann. Rev. Immunol. 18:739-766 (2000), Table 1). Antibodies with substitutions in an Fc region thereof and increased serum half-lives are also described in WO0/42072, WO 02/060919; Shields et al., J. Biol. Chem. 276:6591-6604 (2001); Hinton, J. Biol. Chem. 279:6213-6216 (2004)). In another embodiment, the serum half-life can also be increased, for example, by attaching other polypeptide sequences. For example, antibodies or other polypeptides useful in the methods of the invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one preferred embodiment, the serum albumin peptide to be attached comprises an amino acid sequence of DICLPRWGCLW (SEQ ID NO:37). In another embodiment, the half-life of a Fab is increased by these methods. See also, Dennis et al. J. Biol Chem. 277:35035-35043 (2002) for serum albumin binding peptide sequences.

An “isolated” polypeptide or “isolated” antibody is one that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or nonproteinaceous solutes. In preferred embodiments, the polypeptide or antibody will be purified (1) to greater than 95% by weight of polypeptide or antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide or antibody includes the polypeptide or antibody in situ within recombinant cells since at least one component of the polypeptide's natural environment will not be present. Ordinarily, however, isolated polypeptide or antibody will be prepared by at least one purification step.

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more nucleotides or 10, 20, 30, 40, 50, 60, 70, 80, 90, 100, 120, 140, 160, 180, 190, 200 amino acids or more.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer (such as renal cell carcinoma), prostate cancer, vulval cancer, thyroid cancer, hepatic carcinoma, anal carcinoma, penile carcinoma, testicular cancer, esophagael cancer, tumors of the biliary tract, as well as head and neck cancer.

The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation.

By “dysplasia” is meant any abnormal growth or development of tissue, organ, or cells. Preferably, the dysplasia is high grade or precancerous.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass.

Both stimulatory and inhibitory molecular pathways within the tumor cell regulate this behavior, and interactions between the tumor cell and host cells in the distant site are also significant.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

By “primary tumor” or “primary cancer” is meant the original cancer and not a metastatic lesion located in another tissue, organ, or location in the subject's body.

By “benign tumor” or “benign cancer” is meant a tumor that remains localized at the site of origin and does not have the capacity to infiltrate, invade, or metastasize to a distant site.

By “tumor burden” is meant the number of cancer cells, the size of a tumor, or the amount of cancer in the body. Tumor burden is also referred to as tumor load.

By “tumor number” is meant the number of tumors.

By “subject” is meant a mammal, including, but not limited to, a human or non-human mammal, such as a bovine, equine, canine, ovine, or feline. Preferably, the subject is a human.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets EGFR, PDGFR-beta, BlyS, APRIL, BCMA or VEGF receptor(s), TRAIL/Apo2, and other bioactive and organic chemical agents, etc. Combinations thereof are also included in the invention.

For the purposes herein, “trastuzumab,” “HERCEPTIN®,” and “huMAb4D5-8” refer to an antibody comprising the light and heavy chain amino acid sequences in SEQ ID NOs 13 and 14, respectively.

Herein, “pertuzumab” and “OMNITARG™” and “rhuMab 2C4” refer to an antibody comprising the light and heavy chain amino acid sequences in SEQ ID NO: 3 and 4, respectively. When Pertuzumab is an intact antibody, it preferably comprises the light chain and heavy chain amino acid sequences in SEQ ID NOs: 15 and 16, respectively.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omega1I (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2- ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol- Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified pro drugs, glycosylated prodrugs, β-lactam-containing pro drugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

### Therapeutic Agents

The present invention features the use of c-met antagonists and HER antagonists in combination therapy to treat a pathological condition, such as cancer, in a subject.

### C-Met Antagonists

C-met antagonists useful in the methods of the invention include polypeptides that specifically bind to c-met, anti- c-met antibodies, c-met small molecules, receptor molecules and derivatives which bind specifically to c-met, and fusions proteins. C-met antagonists also include antagonistic variants of c-met polypeptides, RNA aptamers and peptibodies against c-met and HGF. Also included as c-met antagonists useful in the methods of the invention are anti-HGF antibodies, anti-HGF polypeptides, c-met receptor molecules and derivatives which bind specifically to HGF. Examples of each of these are described below.

Anti-c-met antibodies that are useful in the methods of the invention include any antibody that binds with sufficient affinity and specificity to c-met and can reduce or inhibit c-met activity. The antibody selected will normally have a sufficiently strong binding affinity for c-met, for example, the antibody may bind human c-met with a Kd value of between 100 nM-1 pM. Antibody affinities may be determined by a surface plasmon resonance based assay (such as the BlAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein c-met/HGF activity is involved. Also, the antibody may be subjected to other biological activity assays, e.g., in order to evaluate its effectiveness as a therapeutic. Such assays are known in the art and depend on the target antigen and intended use for the antibody.

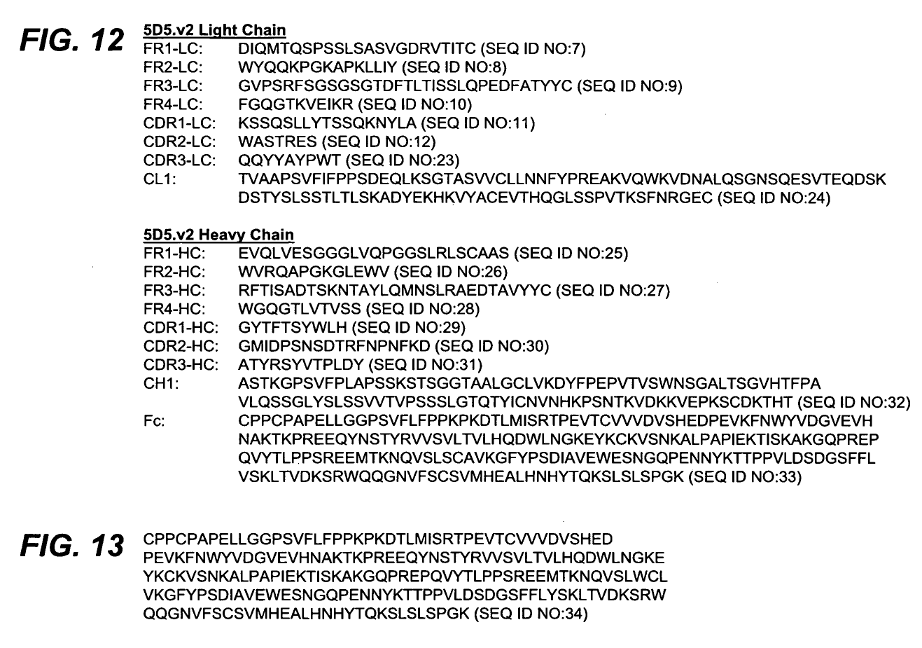

Anti- c-met antibodies are known in the art (see, e.g., Martens, T, et al (2006) Clin Cancer Res 12(20 Pt 1):6144; U.S. Pat. No. 6,468,529; WO2006/015371; WO2007/063816; U.S. Pat. No. 7,408,043; WO2009/007427; WO2005/016382; WO2007/126799. In one embodiment, the anti-c-met antibody comprises a heavy chain variable domain comprising one or more of CDR1-HC, CDR2-HC and CDR3-HC sequence depicted in FIG. 12 (SEQ ID NOs: 29-31). In some embodiments, the antibody comprises a light chain variable domain comprising one or more of CDR1-LC, CDR2-LC and CDR3-LC sequence depicted in FIG. 12 (SEQ ID NOs: 11, 12, 23). In some embodiments, the heavy chain variable domain comprises FR1-HC, FR2-HC, FR3-HC and FR4-HC sequence depicted in FIG. 12 (SEQ ID NOs: 25-28). In some embodiments, the light chain variable domain comprises FR1-LC, FR2-LC, FR3-LC and FR4-LC sequence depicted in FIG. 12 (SEQ ID NOs: 7-10). In some embodiments, the anti-c-met antibody is monovalent and comprises an Fc region. In some embodiments, the antibody comprises Fc sequence depicted in FIG. 12 (SEQ ID NO:33).

In some embodiments, the antibody is monovalent and comprises a Fc region, wherein the Fc region comprises a first and a second polypeptide, wherein the first polypeptide comprises the Fc sequence depicted in FIG. 12 (SEQ ID NO: 33) and the second polypeptide comprises the Fc sequence depicted in FIG. 13 (SEQ ID NO: 34).

In one embodiment, the anti-c-met antibody comprises (a) a first polypeptide comprising a heavy chain variable domain having the sequence:
QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYVSPLDYWGQGTSVTVSS (SEQ ID NO:35), CH1 sequence depicted in FIG. 12, and the Fc sequence depicted in FIG. 12; and (b) a second polypeptide comprising a light chain variable domain having the sequence:
DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ ID NO:36), and CL1 sequence depicted in FIG. 12; and (c) a third polypeptide comprising the Fc sequence depicted in FIG. 13.

In other embodiments, the anti-c-met antibody is the monoclonal antibody produced by the hybridoma cell line deposited under American Type Culture Collection Accession Number ATCC HB-11894 (hybridoma 1A3.3.13) or HB-11895 (hybridoma 5D5.11.6). In other embodiments, the antibody comprises one or more of the CDR sequences of the monoclonal antibody produced by the hybridoma cell line deposited under American Type Culture Collection Accession Number ATCC HB-11894 (hybridoma 1A3.3.13) or HB-11895 (hybridoma 5D5.11.6).

In other embodiments, a c-met antibody of the invention specifically binds at least a portion of c-met Sema domain or variant thereof. In one example, an antagonist antibody of the invention specifically binds at least one of the sequences selected from the group consisting of LDAQT (SEQ ID NO: 38) (e.g., residues 269-273 of c-met), LTEKRKKRS (SEQ ID NO: 39) (e.g., residues 300-308 of c-met), KPDSAEPM (SEQ ID NO: 40) (e.g., residues 350-357 of c-met) and NVRCLQHF (SEQ ID NO: 41) (e.g., residues 381-388 of c-met). In one embodiment, an antagonist antibody of the invention specifically binds a conformational epitope formed by part or all of at least one of the sequences selected from the group consisting of LDAQT (SEQ ID NO: 38) (e.g., residues 269-273 of c-met), LTEKRKKRS (SEQ ID NO: 39) (e.g., residues 300-308 of c-met), KPDSAEPM (SEQ ID NO: 40) (e.g., residues 350-357 of c-met) and NVRCLQHF (SEQ ID NO: 41) (e.g., residues 381-388 of c-met). In one embodiment, an antagonist antibody of the invention specifically binds an amino acid sequence having at least 50%, 60%, 70%, 80%, 90%, 95%, 98% sequence identity or similarity with the sequence LDAQT (SEQ ID NO: 38), LTEKRKKRS (SEQ ID NO: 39), KPDSAEPM (SEQ ID NO: 40) and/or NVRCLQHF (SEQ ID NO: 41).

Anti-HGF antibodies are well known in the art. See, e.g., Kim K J, et al. Clin Cancer Res. (2006) 12(4):1292-8; WO2007/115049; WO2009/002521; WO2007/143098; WO2007/017107; WO2005/017107; L2G7; AMG-102.

C-met receptor molecules or fragments thereof that specifically bind to HGF can be used in the methods of the invention, e.g., to bind to and sequester the HGF protein, thereby preventing it from signaling. Preferably, the c-met receptor molecule, or HGF binding fragment thereof, is a soluble form. In some embodiments, a soluble form of the receptor exerts an inhibitory effect on the biological activity of the c-met protein by binding to HGF, thereby preventing it from binding to its natural receptors present on the surface of target cells. Also included are c-met receptor fusion proteins, examples of which are described below.

A soluble c-met receptor protein or chimeric c-met receptor proteins of the present invention includes c-met receptor proteins which are not fixed to the surface of cells via a transmembrane domain. As such, soluble forms of the c-met receptor, including chimeric receptor proteins, while capable of binding to and inactivating HGF, do not comprise a transmembrane domain and thus generally do not become associated with the cell membrane of cells in which the molecule is expressed. See, e.g., Kong-Beltran, M et al Cancer Cell (2004) 6(1): 75-84.

HGF molecules or fragments thereof that specifically bind to c-met and block or reduce activation of c-met, thereby preventing it from signaling, can be used in the methods of the invention.

Aptamers are nucleic acid molecules that form tertiary structures that specifically bind to a target molecule, such as a HGF polypeptide. The generation and therapeutic use of aptamers are well established in the art. See, e.g., U.S. Pat. No. 5,475,096. A HGF aptamer is a pegylated modified oligonucleotide, which adopts a three-dimensional conformation that enables it to bind to extracellular HGF. Additional information on aptamers can be found in U. S. Patent Application Publication No. 20060148748.

A peptibody is a peptide sequence linked to an amino acid sequence encoding a fragment or portion of an immunoglobulin molecule. Polypeptides may be derived from randomized sequences selected by any method for specific binding, including but not limited to, phage display technology. In a preferred embodiment, the selected polypeptide may be linked to an amino acid sequence encoding the Fc portion of an immunoglobulin. Peptibodies that specifically bind to and antagonize HGF or c-met are also useful in the methods of the invention.

C-met antagonists include small molecules such as compounds described in US 5,792,783; U.S. Pat. No. 5,834,504; U.S. Pat. No. 5,880,141; U.S. Pat. No. 6,297,238; U.S. Pat. No. 6,599,902; U.S. Pat. No. 6,790,852; US 2003/0125370; US 2004/0242603; US 2004/0198750; US 2004/0110758; US 2005/0009845; US 2005/0009840; US 2005/0245547; US 2005/0148574; US 2005/0101650; US 2005/0075340; US 2006/0009453; US 2006/0009493; WO 98/007695; WO 2003/000660; WO 2003/087026; WO 2003/097641; WO 2004/076412; WO 2005/004808; WO 2005/121 125; WO 2005/030140; WO 2005/070891; WO 2005/080393; WO 2006/014325; WO 2006/021886; WO 2006/021881, WO 2007/103308). PHA-665752 is a small molecule, ATP-competitive, active-site inhibitor of the catalytic activity of c-Met, as well as cell growth, cell motility, invasion, and morphology of a variety of tumor cells (Ma et al (2005) Clin. Cancer Res. 11:2312-2319; Christensen et al (2003) Cancer Res. 63:7345-7355).

### HER Antagonists

Anti-HER antibodies that are useful in the methods of the invention include any antibody that binds with sufficient affinity and specificity to HER and can reduce or inhibit HER activity. The antibody selected will normally have a sufficiently strong binding affinity for HER, for example, the antibody may bind human HER with a Kd value of between 100 nM-1 pM. Antibody affinities may be determined by a surface plasmon resonance based assay (such as the BlAcore assay as described in PCT Application Publication No. WO2005/012359); enzyme-linked immunoabsorbent assay (ELISA); and competition assays (e.g. RIA's), for example. Preferably, the anti-HER antibody of the invention can be used as a therapeutic agent in targeting and interfering with diseases or conditions wherein HER/Her ligand activity is involved. Also, the antibody may be subjected to other biological activity assays, e.g., in order to evaluate its effectiveness as a therapeutic. Such assays are known in the art and depend on the target antigen and intended use for the antibody.

Exemplary HER tyrosine kinase inhibitors include small molecule HER2 tyrosine kinase inhibitor such as TAK165 available from Takeda; CP-724,714, an oral selective inhibitor of the ErbB2 receptor tyrosine kinase (Pfizer and OSI); PKI-166 (available from Novartis); pan-HER inhibitors such as canertinib (CI-1033; Pharmacia).

U.S. Pat. No. 6,949,245 describes production of exemplary humanized HER2 antibodies which bind HER2 and block ligand activation of a HER receptor. The humanized antibody of particular interest herein blocks EGF, TGF-α and/or HRG mediated activation of MAPK essentially as effectively as murine monoclonal antibody 2C4 (or a Fab fragment thereof) and/or binds HER2 essentially as effectively as murine monoclonal antibody 2C4 (or a Fab fragment thereof). The humanized antibody herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al, Sequences of Proteins of Immunological Interest, 5th Ed. Public Health Service, National Institutes of Health, Bethesda, Md. (1991). In one embodiment, the humanized antibody comprises FR substitutions at two or all of positions 69H, 71H and 73H.

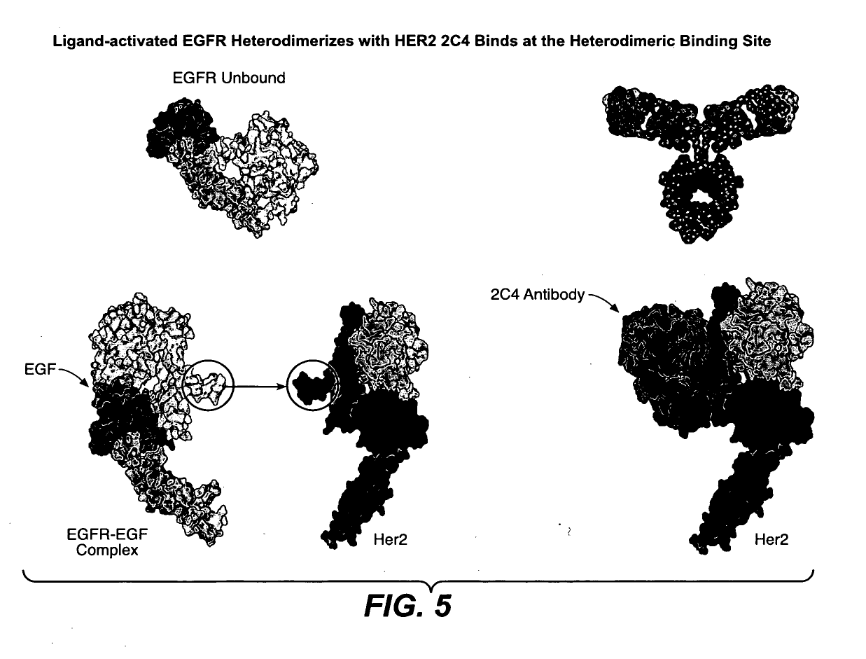

An exemplary humanized antibody of interest herein comprises variable heavy domain complementarity determining residues GFTFTDYTMX, where X is preferably D or S (SEQ ID NO:42); DVNPNSGGSIYNQRFKG (SEQ ID NO:43); and/or NLGPSFYFDY (SEQ ID NO:44), optionally comprising amino acid modifications of those CDR residues, e.g. where the modifications essentially maintain or improve affinity of the antibody. For example, the antibody variant of interest may have from about one to about seven or about five amino acid substitutions in the above variable heavy CDR sequences. Such antibody variants may be prepared by affinity maturation, e.g., as described below. An exemplary humanized antibody comprises the variable heavy domain amino acid sequence in FIG. 5B.

The humanized antibody may comprise variable light domain complementarity determining residues KASQDVSIGVA (SEQ ID NO: 45); SASYX¹X²X³, where X¹ is preferably R or L, X² is preferably Y or E, and X³ is preferably T or S (SEQ ID NO:46); and/or QQYYIYPYT (SEQ ID NO:47), e.g. in addition to those variable heavy domain CDR residues in the preceding paragraph. Such humanized antibodies optionally comprise amino acid modifications of the above CDR residues, e.g. where the modifications essentially maintain or improve affinity of the antibody. For example, the antibody variant of interest may have from about one to about seven or about five amino acid substitutions in the above variable light CDR sequences. Such antibody variants may be prepared by affinity maturation, e.g., as described below. An exemplary humanized antibody comprises the variable light domain amino acid sequence in FIG. 5A.

The present application also contemplates affinity matured antibodies which bind HER2 and block ligand activation of a HER receptor. The parent antibody may be a human antibody or a humanized antibody, e.g., one comprising the variable light and/or variable heavy sequences of FIGS. 5A and 5B, respectively (i.e. comprising the VL and/or VH of pertuzumab). The affinity matured antibody preferably binds to HER2 receptor with an affinity superior to that of murine 2C4 or pertuzumab (e.g. from about two or about four fold, to about 100 fold or about 1000 fold improved affinity, e.g. as assessed using a HER2-extracellular domain (ECD) ELISA). Exemplary variable heavy CDR residues for substitution include H28, H30, H34, H35, H64, H96, H99, or combinations of two or more (e.g. two, three, four, five, six, or seven of these residues). Examples of variable light CDR residues for alteration include L28, L50, L53, L56, L91, L92, L93, L94, L96, L97 or combinations of two or more (e.g. two to three, four, five or up to about ten of these residues).

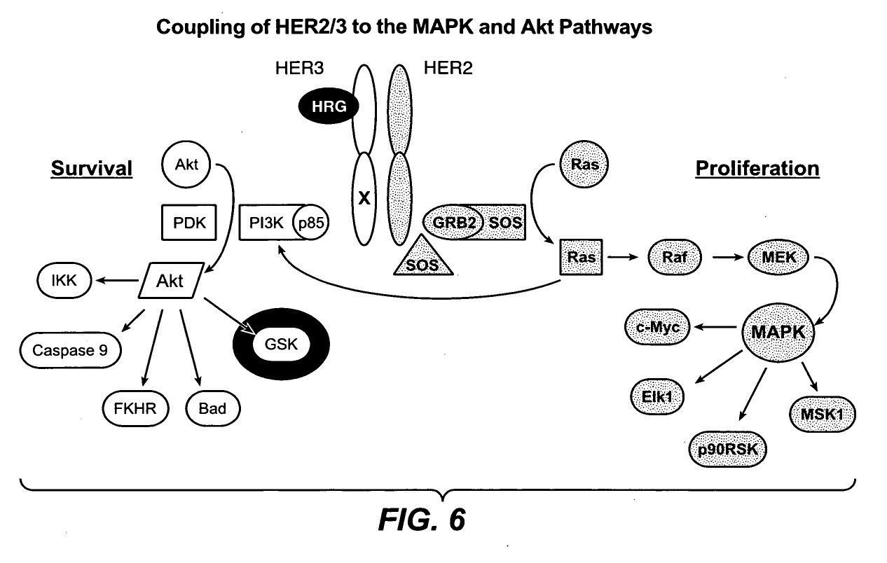

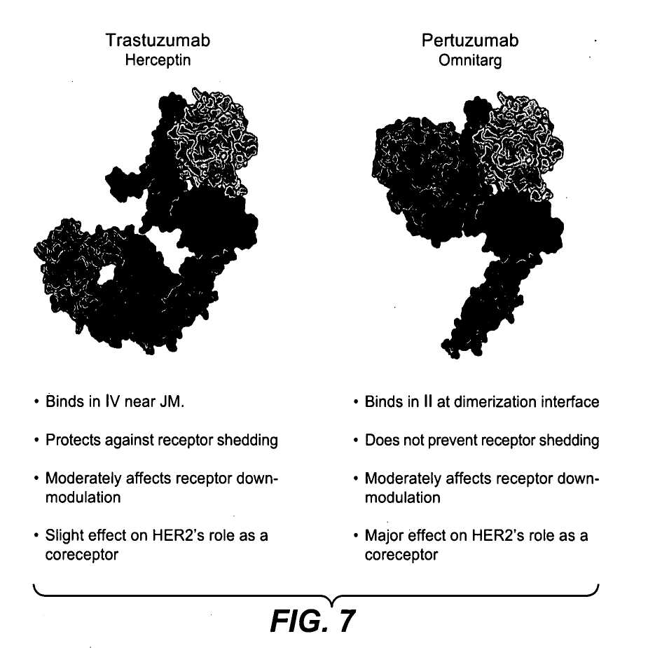

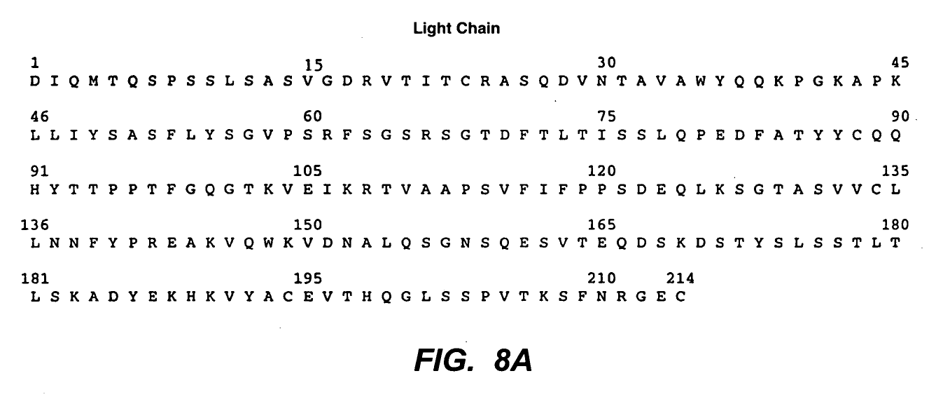

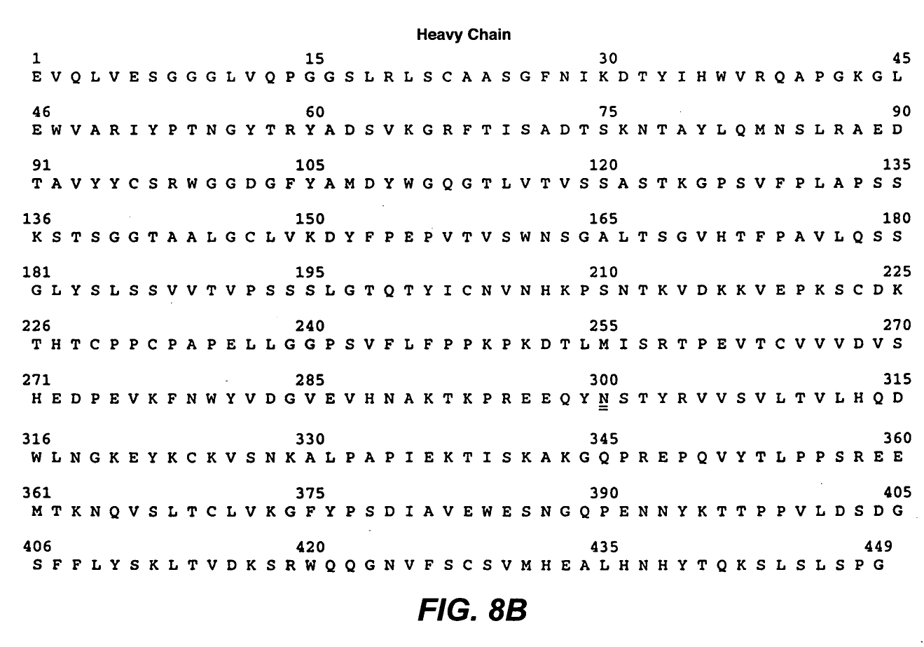

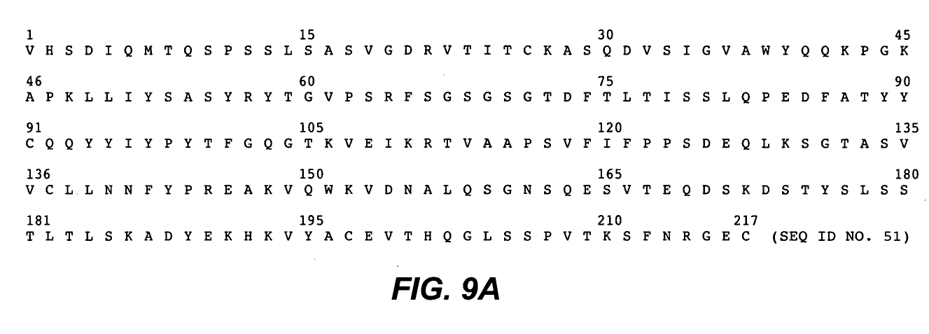

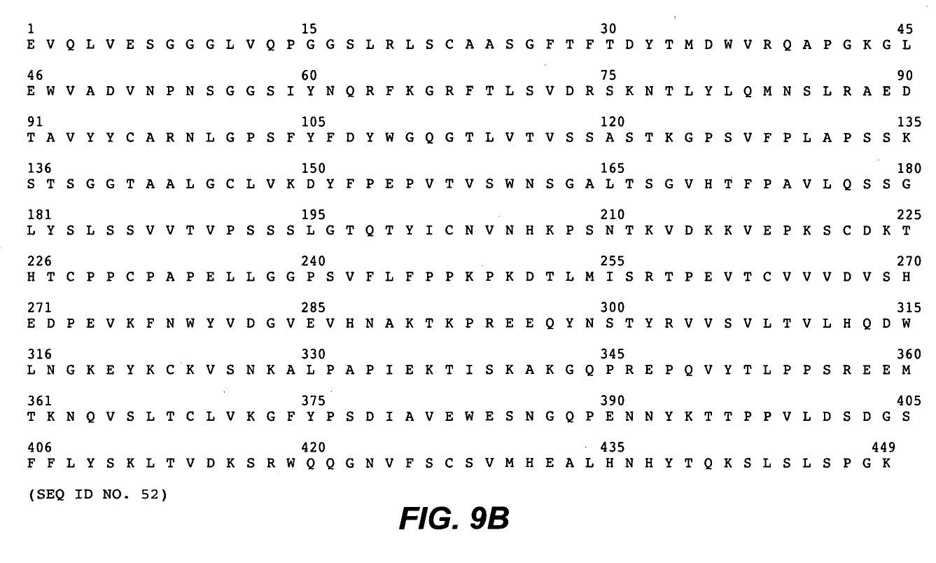

Various forms of the humanized antibody or affinity matured antibody are contemplated. For example, the humanized antibody or affinity matured antibody may be an antibody fragment, such as a Fab, which is optionally conjugated with one or more cytotoxic agent(s) in order to generate an immunoconjugate. Alternatively, the humanized antibody or affinity matured antibody may be an intact antibody, such as an intact IgGi antibody. An exemplary intact IgG1 antibody comprises the light chain sequence in FIG. 6A and the heavy chain sequence in FIG. 6B.

Human HER2 antibodies are described in U.S. Pat. No. 5,772,997 issued Jun. 30, 1998 and WO 97/00271 published Jan. 3, 1997.

Bispecific antibodies are antibodies that have binding specificities for at least two different epitopes. Exemplary bispecific antibodies may bind a HER protein and to c-met. In another example, an exemplary bispecific antibody may bind to two different epitopes of the HER2 protein. Other such antibodies may combine a HER2 binding site with binding site(s) for HER3. Alternatively, a HER2 arm may be combined with an arm which binds to a triggering molecule on a leukocyte such as a T-cell receptor molecule (e.g. CD2 or CD3), or Fc receptors for IgG (FcγR), such as FcγR1 (CD64), FcγRII (CD32) and FcγRIII (CD16) so as to focus cellular defense mechanisms to the HER2-expressing cell. Bispecific antibodies may also be used to localize cytotoxic agents to cells which express HER2. These antibodies possess a HER2-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂bispecific antibodies).

WO 96/16673 describes a bispecific HER2/FcγRIII antibody and U.S. Pat. No. 5,837,234 discloses a bispecific HER2/FcγRI antibody IDM1 (Osidem). A bispecific HER2/Fcα antibody is shown in WO98/02463. U.S. Pat. No. 5,821,337 teaches a bispecific HER2/CD3 antibody. MDX-210 is a bispecific HER2-FcγRIII Ab.

### Combination Therapies

The present invention features the combination use of a c-met antagonist and an HER antagonist as part of a specific treatment regimen intended to provide a beneficial effect from the combined activity of these therapeutic agents. The beneficial effect of the combination includes, but is not limited to, pharmacokinetic or pharmacodynamic co-action resulting from the combination of therapeutic agents. The present invention is particularly useful in treating cancers of various types at various stages.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from the original site and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade, and cancer cells are described as being well-differentiated (low grade), moderately-differentiated, poorly-differentiated, or undifferentiated (high grade). Well-differentiated cells are quite normal appearing and resemble the normal cells from which they originated. Undifferentiated cells are cells that have become so abnormal that it is no longer possible to determine the origin of the cells.

Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

To stage cancer, the American Joint Committee on Cancer first places the cancer, particularly solid tumors, in a letter category using the TNM classification system. Cancers are designated the letter T (tumor size), N (palpable nodes), and/or M (metastases). T1, T2, T3, and T4 describe the increasing size of the primary lesion; N0, N1, N2, N3 indicates progressively advancing node involvement; and M0 and M1 reflect the absence or presence of distant metastases.

In the second staging method, also known as the Overall Stage Grouping or Roman Numeral Staging, cancers are divided into stages 0 to IV, incorporating the size of primary lesions as well as the presence of nodal spread and of distant metastases. In this system, cases are grouped into four stages denoted by Roman numerals I through IV, or are classified as “recurrent.” For some cancers, stage 0 is referred to as “in situ” or “Tis,” such as ductal carcinoma in situ or lobular carcinoma in situ for breast cancers. High grade adenomas can also be classified as stage 0. In general, stage I cancers are small localized cancers that are usually curable, while stage IV usually represents inoperable or metastatic cancer. Stage II and III cancers are usually locally advanced and/or exhibit involvement of local lymph nodes. In general, the higher stage numbers indicate more extensive disease, including greater tumor size and/or spread of the cancer to nearby lymph nodes and/or organs adjacent to the primary tumor. These stages are defined precisely, but the definition is different for each kind of cancer and is known to the skilled artisan.

Many cancer registries, such as the NCI's Surveillance, Epidemiology, and End Results Program (SEER), use summary staging. This system is used for all types of cancer. It groups cancer cases into five main categories:

In situ is early cancer that is present only in the layer of cells in which it began.

Localized is cancer that is limited to the organ in which it began, without evidence of spread.

Regional is cancer that has spread beyond the original (primary) site to nearby lymph nodes or organs and tissues.

Distant is cancer that has spread from the primary site to distant organs or distant lymph nodes.

Unknown is used to describe cases for which there is not enough information to indicate a stage.

In addition, it is common for cancer to return months or years after the primary tumor has been removed. Cancer that recurs after all visible tumor has been eradicated, is called recurrent disease. Disease that recurs in the area of the primary tumor is locally recurrent, and disease that recurs as metastases is referred to as a distant recurrence.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors. Other examples of cancers are provided in the Definitions.

In some embodiments, the patient herein is subjected to a diagnostic test e.g., prior to and/or during and/or after therapy. Generally, if a diagnostic test is performed, a sample may be obtained from a patient in need of therapy. Where the subject has cancer, the sample may be a tumor sample, or other biological sample, such as a biological fluid, including, without limitation, blood, urine, saliva, ascites fluid, or derivatives such as blood serum and blood plasma, and the like.

In some embodiments, the cancer is HER positive, such that, e.g., a HER antibody is able to bind to the cancer cells. In one embodiment, the cancer expresses low HER3 (e.g., ovarian cancer) or has elevated HER2:3 ratio (e.g. ovarian cancer). In some embodiments, the cancer is c-met positive, such that, e.g., a c-met antibody is able to bind to the cancer cells.

The biological sample herein may be a fixed sample, e.g. a formalin fixed, paraffin-embedded (FFPE) sample, or a frozen sample.

Various methods for determining expression of mRNA or protein include, but are not limited to, gene expression profiling, polymerase chain reaction (PCR) including quantitative real time PCR (qRT-PCR), microarray analysis, serial analysis of gene expression (SAGE), MassARRAY, Gene Expression Analysis by Massively Parallel Signature Sequencing (MPSS), proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR). In one embodiment, expression of one or more of the above noted genes is deemed positive expression if it is at the median or above, e.g. compared to other samples of the same tumor-type. The median expression level can be determined essentially contemporaneously with measuring gene expression, or may have been determined previously.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

Detection of gene or protein expression may be determined directly or indirectly.

One may determine expression or amplification of c-met and/or HER in the cancer (directly or indirectly). Various diagnostic/prognostic assays are available for this. In one embodiment, c-met and/or HER overexpression may be analyzed by IHC. Paraffin embedded tissue sections from a tumor biopsy may be subjected to the IHC assay and accorded a c-met and/or HER protein staining intensity criteria as follows:

Score 0 no staining is observed or membrane staining is observed in less than 10% of tumor cells.

Score 1+ a faint/barely perceptible membrane staining is detected in more than 10% of the tumor cells. The cells are only stained in part of their membrane.

Score 2+ a weak to moderate complete membrane staining is observed in more than 10% of the tumor cells.

Score 3+ a moderate to strong complete membrane staining is observed in more than 10% of the tumor cells.

In some embodiments, those tumors with 0 or 1+ scores for c-met and/or HER overexpression assessment may be characterized as not overexpressing c-met and/or HER, whereas those tumors with 2+ or 3+ scores may be characterized as overexpressing c-met and/or HER.

In some embodiments, tumors overexpressing c-met and/or HER may be rated by immunohistochemical scores corresponding to the number of copies of c-met and/or HER molecules expressed per cell, and can been determined biochemically:

0=0-10,000 copies/cell,

1+=at least about 200,000 copies/cell,

2+=at least about 500,000 copies/cell,

3+=at least about 2,000,000 copies/cell.

Alternatively, or additionally, FISH assays may be carried out on formalin-fixed, paraffin-embedded tumor tissue to determine the extent (if any) of c-met and/or HER amplification in the tumor.

C-met or HER activation may be determined directly (e.g., by phosph-ELISA testing, or other means of detecting phosphorylated receptor) or indirectly (e.g., by detection of activated downstream signaling pathway components, detection of receptor dimers (e.g., homodimers, heterodimers), detection of gene expression profiles and the like.

Similarly, c-met or HER constitutive activation or presence of ligand-independent HER or c-met may be detected directly or indirectly (e.g., by detection of receptor mutations correlated with constitutive activity, by detection of receptor amplification correlated with constitutive activity and the like).

Methods for detection of nucleic acid mutations are well known in the art. Often, though not necessarily, a target nucleic acid in a sample is amplified to provide the desired amount of material for determination of whether a mutation is present. Amplification techniques are well known in the art. For example, the amplified product may or may not encompass all of the nucleic acid sequence encoding the protein of interest, so long as the amplified product comprises the particular amino acid/nucleic acid sequence position where the mutation is suspected to be.

In one example, presence of a mutation can be determined by contacting nucleic acid from a sample with a nucleic acid probe that is capable of specifically hybridizing to nucleic acid encoding a mutated nucleic acid, and detecting said hybridization. In one embodiment, the probe is detectably labeled, for example with a radioisotope (³H, ³²P, ³³P etc), a fluorescent agent (rhodamine, fluorescene etc.) or a chromogenic agent. In some embodiments, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, nucleic acid probes of the invention are provided in a kit for identifying c-met mutations in a sample, said kit comprising an oligonucleotide that specifically hybridizes to or adjacent to a site of mutation in the nucleic acid encoding, e.g., c-met. The kit may further comprise instructions for treating patients having tumors that contain, e.g., c-met mutations with a c-met antagonist based on the result of a hybridization test using the kit.

Mutations can also be detected by comparing the electrophoretic mobility of an amplified nucleic acid to the electrophoretic mobility of corresponding nucleic acid encoding wild-type c-met. A difference in the mobility indicates the presence of a mutation in the amplified nucleic acid sequence. Electrophoretic mobility may be determined by any appropriate molecular separation technique, for example on a polyacrylamide gel.

Nucleic acids may also be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from nucleic acid alterations such as point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from amplification reactions, eliminating the need for sample purification, shortening the hybridization time, and increasing the signal-to-noise ratio. Mixed samples containing up to a 20-fold excess of normal nucleic acids and fragments up to 4 kb in size can been assayed. However, EMD scanning does not identify particular base changes that occur in mutation positive samples, therefore often requiring additional sequencing procedures to identify the specific mutation if necessary. CEL I enzyme can be used similarly to resolvase T₄ endonuclease VII, as demonstrated in U.S. Pat. No. 5,869,245.

Another simple kit for detecting mutations is a reverse hybridization test strip similar to Haemochromatosis StripAssay™ (Viennalabs http://www.bamburghmarrsh.com/pdf/4220.pdf) for detection of multiple mutations in HFE, TFR2 and FPN1 genes causing Haemochromatosis. Such an assay is based on sequence specific hybridization following amplification by PCR. For single mutation assays, a microplate-based detection system may be applied, whereas for multi-mutation assays, test strips may be used as “macro-arrays”. Kits may include ready-to use reagents for sample prep, amplification and mutation detection. Multiplex amplification protocols provide convenience and allow testing of samples with very limited volumes. Using the straightforward StripAssay format, testing for twenty and more mutations may be completed in less than five hours without costly equipment. DNA is isolated from a sample and the target nucleic acid is amplified in vitro (e.g., by PCR) and biotin-labeled, generally in a single (“multiplex”) amplification reaction. The amplification products are then selectively hybridized to oligonucleotide probes (wild-type and mutant specific) immobilized on a solid support such as a test strip in which the probes are immobilized as parallel lines or bands. Bound biotinylated amplicons are detected using streptavidin-alkaline phosphatase and color substrates. Such an assay can detect all or any subset of the mutations of the invention. With respect to a particular mutant probe band, one of three signaling patterns are possible: (i) a band only for wild-type probe which indicates normal nucleic acid sequence, (ii) bands for both wild-type and a mutant probe which indicates heterozygous genotype, and (iii) band only for the mutant probe which indicates homozygous mutant genotype. Accordingly, in one aspect, the invention provides a method of detecting mutations of the invention comprising isolating and/or amplifying a target c-met nucleic acid sequence from a sample, such that the amplification product comprises a ligand, contacting the amplification product with a probe which comprises a detectable binding partner to the ligand and the probe is capable of specifically hybridizing to a mutation of the invention, and then detecting the hybridization of said probe to said amplification product. In one embodiment, the ligand is biotin and the binding partner comprises avidin or streptavidin. In one embodiment, the binding partner comprises steptavidin-alkaline which is detectable with color substrates. In one embodiment, the probes are immobilized for example on a test strip wherein probes complementary to different mutations are separated from one another. Alternatively, the amplified nucleic acid is labeled with a radioisotope in which case the probe need not comprise a detectable label.

Alterations of a wild-type gene encompass all forms of mutations such as insertions, inversions, deletions, and/or point mutations. In one embodiment, the mutations are somatic. Somatic mutations are those which occur only in certain tissues, e.g., in the tumor tissue, and are not inherited in the germ line. Germ line mutations can be found in any of a body's tissues.

A sample comprising a target nucleic acid can be obtained by methods well known in the art, and that are appropriate for the particular type and location of the tumor. Tissue biopsy is often used to obtain a representative piece of tumor tissue. Alternatively, tumor cells can be obtained indirectly in the form of tissues/fluids that are known or thought to contain the tumor cells of interest. For instance, samples of lung cancer lesions may be obtained by resection, bronchoscopy, fine needle aspiration, bronchial brushings, or from sputum, pleural fluid or blood. Mutant genes or gene products can be detected from tumor or from other body samples such as urine, sputum or serum. The same techniques discussed above for detection of mutant target genes or gene products in tumor samples can be applied to other body samples. Cancer cells are sloughed off from tumors and appear in such body samples. By screening such body samples, a simple early diagnosis can be achieved for diseases such as cancer. In addition, the progress of therapy can be monitored more easily by testing such body samples for mutant target genes or gene products.

Means for enriching a tissue preparation for tumor cells are known in the art. For example, the tissue may be isolated from paraffin or cryostat sections. Cancer cells may also be separated from normal cells by flow cytometry or laser capture microdissection. These, as well as other techniques for separating tumor from normal cells, are well known in the art. If the tumor tissue is highly contaminated with normal cells, detection of mutations may be more difficult, although techniques for minimizing contamination and/or false positive/negative results are known, some of which are described hereinbelow. For example, a sample may also be assessed for the presence of a biomarker (including a mutation) known to be associated with a tumor cell of interest but not a corresponding normal cell, or vice versa.

Detection of point mutations in target nucleic acids may be accomplished by molecular cloning of the target nucleic acids and sequencing the nucleic acids using techniques well known in the art. Alternatively, amplification techniques such as the polymerase chain reaction (PCR) can be used to amplify target nucleic acid sequences directly from a genomic DNA preparation from the tumor tissue. The nucleic acid sequence of the amplified sequences can then be determined and mutations identified therefrom. Amplification techniques are well known in the art, e.g., polymerase chain reaction as described in Saiki et al., Science 239:487, 1988; U.S. Pat. Nos. 4,683,203 and 4,683,195.

It should be noted that design and selection of appropriate primers are well established techniques in the art.

The ligase chain reaction, which is known in the art, can also be used to amplify target nucleic acid sequences. See, e.g., Wu et al., Genomics, Vol. 4, pp. 560-569 (1989). In addition, a technique known as allele specific PCR can also be used. See, e.g., Ruano and Kidd, Nucleic Acids Research, Vol. 17, p. 8392, 1989. According to this technique, primers are used which hybridize at their 3′ends to a particular target nucleic acid mutation. If the particular mutation is not present, an amplification product is not observed. Amplification Refractory Mutation System (ARMS) can also be used, as disclosed in European Patent Application Publication No. 0332435, and in Newton et al., Nucleic Acids Research, Vol. 17, p. 7, 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Single stranded conformation polymorphism (SSCP) analysis can also be used to detect base change variants of an allele. See, e.g. Orita et al., Proc. Natl. Acad. Sci. USA Vol. 86, pp. 2766-2770, 1989, and Genomics, Vol. 5, pp. 874-879, 1989. Other techniques for detecting insertions and deletions as known in the art can also be used.

Alteration of wild-type genes can also be detected on the basis of the alteration of a wild-type expression product of the gene. Such expression products include both mRNA as well as the protein product. Point mutations may be detected by amplifying and sequencing the mRNA or via molecular cloning of cDNA made from the MRNA. The sequence of the cloned cDNA can be determined using DNA sequencing techniques which are well known in the art. The cDNA can also be sequenced via the polymerase chain reaction (PCR).

Mismatches are hybridized nucleic acid duplexes which are not 100% complementary. The lack of total complementarity may be due to deletions, insertions, inversions, substitutions or frameshift mutations. Mismatch detection can be used to detect point mutations in a target nucleic acid. While these techniques can be less sensitive than sequencing, they are simpler to perform on a large number of tissue samples. An example of a mismatch cleavage technique is the RNase protection method, which is described in detail in Winter et al., Proc. Natl. Acad. Sci. USA, Vol. 82, p. 7575, 1985, and Meyers et al., Science, Vol. 230, p. 1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid mRNA or gene, but can a portion of the target nucleic acid, provided it encompasses the position suspected of being mutated. If the riboprobe comprises only a segment of the target nucleic acid mRNA or gene, it may be desirable to use a number of these probes to screen the whole target nucleic acid sequence for mismatches if desired.

In a similar manner, DNA probes can be used to detect mismatches, for example through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the target nucleic acid mRNA or DNA which might contain a mutation can be amplified before hybridization. Changes in target nucleic acid DNA can also be detected using Southern hybridization, especially if the changes are gross rearrangements, such as deletions and insertions.

Target nucleic acid DNA sequences which have been amplified may also be screened using allele-specific probes. These probes are nucleic acid oligomers, each of which contains a region of the target nucleic acid gene harboring a known mutation. For example, one oligomer may be about 30 nucleotides in length, corresponding to a portion of the target gene sequence. By use of a battery of such allele-specific probes, target nucleic acid amplification products can be screened to identify the presence of a previously identified mutation in the target gene. Hybridization of allele-specific probes with amplified target nucleic acid sequences can be performed, for example, on a nylon filter. Hybridization to a particular probe under stringent hybridization conditions indicates the presence of the same mutation in the tumor tissue as in the allele-specific probe.

Alteration of wild-type target genes can also be detected by screening for alteration of the corresponding wild-type protein. For example, monoclonal antibodies immunoreactive with a target gene product can be used to screen a tissue, for example an antibody that is known to bind to a particular mutated position of the gene product (protein). For example, an antibody that is used may be one that binds to a deleted exon (e.g., exon 14) or that binds to a conformational epitope comprising a deleted portion of the target protein. Lack of cognate antigen would indicate a mutation. Antibodies specific for products of mutant alleles could also be used to detect mutant gene product. Antibodies may be identified from phage display libraries. Such immunological assays can be done in any convenient format known in the art. These include Western blots, immunohistochemical assays and ELISA assays. Any means for detecting an altered protein can be used to detect alteration of wild-type target genes.

Primer pairs are useful for determination of the nucleotide sequence of a target nucleic acid using nucleic acid amplification techniques such as the polymerase chain reaction. The pairs of single stranded DNA primers can be annealed to sequences within or surrounding the target nucleic acid sequence in order to prime amplification of the target sequence. Allele-specific primers can also be used. Such primers anneal only to particular mutant target sequence, and thus will only amplify a product in the presence of the mutant target sequence as a template. In order to facilitate subsequent cloning of amplified sequences, primers may have restriction enzyme site sequences appended to their ends. Such enzymes and sites are well known in the art. The primers themselves can be synthesized using techniques which are well known in the art. Generally, the primers can be made using oligonucleotide synthesizing machines which are commercially available. Design of particular primers is well within the skill of the art.

Nucleic acid probes are useful for a number of purposes. They can be used in Southern hybridization to genomic DNA and in the RNase protection method for detecting point mutations already discussed above. The probes can be used to detect target nucleic acid amplification products. They may also be used to detect mismatches with the wild type gene or mRNA using other techniques. Mismatches can be detected using either enzymes (e.g., SI nuclease), chemicals (e.g., hydroxylamine or osmium tetroxide and piperidine), or changes in electrophoretic mobility of mismatched hybrids as compared to totally matched hybrids. These techniques are known in the art. See Novack et al., Proc. Natl. Acad. Sci. USA, Vol. 83, p. 586, 1986. Generally, the probes are complementary to sequences outside of the kinase domain. An entire battery of nucleic acid probes may be used to compose a kit for detecting mutations in target nucleic acids. The kit allows for hybridization to a large region of a target sequence of interest. The probes may overlap with each other or be contiguous.

If a riboprobe is used to detect mismatches with mRNA, it is generally complementary to the mRNA of the target gene. The riboprobe thus is an antisense probe in that it does not code for the corresponding gene product because it is complementary to the sense strand. The riboprobe generally will be labeled with a radioactive, colorimetric, or fluorometric material, which can be accomplished by any means known in the art. If the riboprobe is used to detect mismatches with DNA it can be of either polarity, sense or anti-sense. Similarly, DNA probes also may be used to detect mismatches.

In some instances, the cancer does or does not overexpress c-met and/or HER. Receptor overexpression may be determined in a diagnostic or prognostic assay by evaluating increased levels of the receptorprotein present on the surface of a cell (e.g. via an immunohistochemistry assay; IHC). Alternatively, or additionally, one may measure levels of receptor-encoding nucleic acid in the cell, e.g. via fluorescent in situ hybridization (FISH; see WO98/45479 published October, 1998), southern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

In some instances, the invention provides methods for reducing growth and/or proliferation of a cancer cell, or increasing apoptosis of a cancer cell. Methods for examining growth and/or proliferation of a cancer cell, or increasing apoptosis of a cancer cell are well known in the art and some are described and exemplified herein. Exemplary methods for determining cell grown and/or proliferation and/or apoptosis include, for example, BrdU incorporation assay, MTT, [³H]-thymidine incorporation (e.g., TopCount assay (PerkinElmer)), cell viability assays (e.g., CellTiter-Glo (Promega)), DNA fragmentation assays, caspase activation assays, tryptan blue exclusion, chromatin morphology assays and the like.

In some instances, the invention provides methods for restoring the sensitivity of a cancer ell to an ErbB antagonist (e.g., an HER antagonist), reducing resistance of a cancer cell to an ErbB antagonist (such as an HER antagonist), and/or treating acquired ErbB antagonist (such as an HER antagonist) resistance in a cancer cell. Methods for examining cell sensitivity and/or resistance to an ErbB antagonist (e.g., an HER antagonist) and/or resistance to an ErbB antagonist are known in the art and some are described herein. For example, the amount of cell growth and/or proliferation and/or amount of apoptosis may be determined, for example, in the presence of the ErbB antagonist. In other embodiments, the amount of cell growth and/or proliferation and/or amount of apoptosis may be determined in the presence of ErbB/c-met antagonist combination treatment as compared to the ErbB antagonist treatment alone.

In some instances, the invention provides methods for reducing PI3K (phosphoinositide-3 kinase) mediated signaling in a cancer cell. Methods for examining PI3K mediated signaling are known in the art and some methods are disclosed and exemplified herein. In some embodiments, the presence or absence of phosphorylated forms of proteins that are phosphorylated in response to PI3K activation (e.g., Akt) can be assayed using immunoassays.

### Chemotherapeutic Agents

The combination therapy of the invention can further comprise one or more chemotherapeutic agent(s). The combined administration includes coadministration or concurrent administration, using separate formulations or a single pharmaceutical formulation, and consecutive administration in either order, wherein preferably there is a time period while both (or all) active agents simultaneously exert their biological activities.

The chemotherapeutic agent, if administered, is usually administered at dosages known therefor, or optionally lowered due to combined action of the drugs or negative side effects attributable to administration of the antimetabolite chemotherapeutic agent. Preparation and dosing schedules for such chemotherapeutic agents may be used according to manufacturers' instructions or as determined empirically by the skilled practitioner.

Various chemotherapeutic agents that can be combined are disclosed above. Preferred chemotherapeutic agents to be combined are selected from the group consisting of a taxoid (including docetaxel and paclitaxel), vinca (such as vinorelbine or vinblastine), platinum compound (such as carboplatin or cisplatin), aromatase inhibitor (such as letrozole, anastrazole, or exemestane), anti-estrogen (e.g. fulvestrant or tamoxifen), etoposide, thiotepa, cyclophosphamide, methotrexate, liposomal doxorubicin, pegylated liposomal doxorubicin, capecitabine, gemcitabine, COX-2 inhibitor (for instance, celecoxib), or proteosome inhibitor (e.g. PS342).

### Formulations, Dosages and Administrations

The therapeutic agents used in the invention will be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, the drug-drug interaction of the agents to be combined, and other factors known to medical practitioners.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENT™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The therapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In the case of VEGF antagonists, local administration is particularly desired if extensive side effects or toxicity is associated with VEGF antagonism. An ex vivo strategy can also be used for therapeutic applications. Ex vivo strategies involve transfecting or transducing cells obtained from the subject with a polynucleotide encoding a c-met or HER antagonist. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

For example, if the c-met or HER antagonist is an antibody, the antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Preferably the dosing is given by injections, most preferably intravenous or subcutaneous injections, depending in part on whether the administration is brief or chronic.

In another example, the c-met or HER antagonist compound is administered locally, e.g., by direct injections, when the disorder or location of the tumor permits, and the injections can be repeated periodically. The c-met or HER antagonist can also be delivered systemically to the subject or directly to the tumor cells, e.g., to a tumor or a tumor bed following surgical excision of the tumor, in order to prevent or reduce local recurrence or metastasis.

Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, the HER or c-met antagonist in the combination may be administered by intravenous injection while the protein kinase inhibitor in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

Depending on the type and severity of the disease, about 1 μg/kg to 100 mg/kg (e.g., 0.1-20 mg/kg) of each therapeutic agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above. However, other dosage regimens may be useful. In one example, if the cmet or HER antagonist is an antibody, the antibody of the invention is administered every two to three weeks, at a dose ranging from about 5 mg/kg to about 15 mg/kg. If the c-met or HER antagonist is an oral small molecule compound, the drug may be administered daily at a dose ranging from about 25 mg/kg to about 50 mg/kg. Moreover, the oral compound of the invention can be administered either under a traditional high-dose intermittent regimen, or using lower and more frequent doses without scheduled breaks (referred to as “metronomic therapy”). When an intermittent regimen is used, for example, the drug can be given daily for two to three weeks followed by a one week break; or daily for four weeks followed by a two week break, depending on the daily dose and particular indication. The progress of the therapy of the invention is easily monitored by conventional techniques and assays.

The present application contemplates administration of the c-met and/or HER antagonist by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

The following are examples of the methods and compositions of the invention. It is understood that various other embodiments may be practiced, given the general description provided above.

### EXAMPLES

### Example 1

### Reduction of C-Met Protein Expression in NSCLC Cells Increases Ligand-Induced Activation of EGFR Family of Receptors

### Materials and Methods

Retroviral shRNA constructs. Oligonucleotides coding shRNA sequences against c-met (5′-GATCCCCGAACAGAATCACTGACATATTCAAGAGATATGTCAGTGATTCTGTTCTTTT TTGGAAA-3′ (SEQ ID NO:48) (shMet 3) and 5′ GATCCCCGAAACTGTATGCTGGATGATTCAAGAGATCATCCAGCATACAGTTTCTTT TTTGGAAA (SEQ ID NO:49) (shMet 4)) were cloned into BglII/HindIII sites of the pShuttle-H1 vector downstream of the H1 promoter (David Davis, GNE). BOLD text signifies the target hybridizing sequence. These constructs were recombined with the retroviral pHUSH-GW vector (Gray D et al BMC Biotechnology. 2007; 7:61) using Clonase II enzyme (Invitrogen), generating a construct in which shRNA expression is under control of an inducible promoter. Treatment with the tetracycline analog doxycycline results in shRNA expression. The shGFP2 control retroviral construct containing a shRNA directed against GFP (Hoeflich et al. Cancer Res. (2006) 66(2):999-1006) was provided by David Davis, Genentech, Inc. shGFP2 contains the following oligonucleotide:

(EGFP) shRNA

(SEQ ID NO:50)

(sense) 5′-GATCCCCAGATCCGCCACAACATCGATTCAAGAGATCGA
TGTTGTGGCGGATCTTGTTTTTTGGAAA-3.

Cell Culture. GP-293 packaging cells (Clontech) were maintained in HGDMEM (GNE) supplemented with 10% Tet-Free FBS (Clontech), 2 mM L-Glutamine (GNE), and 100 U/ml penicillin and 100 U/ml streptomycin (Gibco). H441 cells (ATCC No. HTB-174) were maintained in 50:50 media (DMEM:F12, MediaTech) supplemented with 10% Tet-Free FBS (Clontech), 2 mM L-Glutamine (GNE), and 100 U/ml penicillin and 100 U/ml streptomycin (Gibco). EBC-1 cells (Japanese Health Sciences Resources; see Cancer Res. (2005) 65(16):7276-82) were maintained in RPMI 1640 (GNE) supplemented with 10% Tet-Free FBS (Clontech), 2 mM L-Glutamine (GNE), and 100 U/ml penicillin and 100 U/ml streptomycin (Gibco). Cells were maintained at 37° C. with 5% CO2.

Development of recombinant retrovirus and stable lines. GP-293 packaging cells were cotransfected using FuGene 6 (Roche) and CalPhos Mammalian Transfection kit (Clontech) with pVSV-G (Clontech) and the above recombinant retroviral constructs. Media containing the recombinant virus was then added to EBC-1 and H441 cells and cells were selected in Puromycin (Clontech). Cells stably expressing retroviral constructs were then autocloned via FACS into 96 well plates.

Western blot. To resolve proteins, 20 ug of whole cell lysate was run on 4-12% Bis-Tris NuPAGE gel with MOPS buffer (Invitrogen). Gels were equilibrated in 2× NUPAGE transfer buffer with anti-oxidant buffer then transferred to 0.2 um PVDF membrane by iBlot. Membranes were blocked in TBST (10 mM TRIS, pH 8.0, 150 mM NaCl, 0.1% Tween 20) containing 5% BSA for one hour at room temperature then incubated overnight in primary antibody diluted in blocking buffer at 4° C. Membranes were washed with TBST then incubated with the HRP-conjugated secondary antibody (GE Healthcare) in TBST with 5% nonfat milk for one hour at room temperature. Antibodies were detected by chemiluminescence (GE Healthcare, ECL Plus).

Screening of stable cell lines. Clones stably transduced with retroviral constructs were grown in the appropriate media ±1 μg/ml doxycycline (Clontech) to induce expression of the shRNA, and screened via western blots for c-met knockdown using anti-c-met C-12 antibody (Santa Cruz Biotech). Phospho-c-met was blotted for using anti-Phospho-c-met Y1003 (Biosource) and anti-Phospho-c-met Y1234/1234 (Cell Signaling) antibodies. As a control, actin was blotted for using anti-Actin 1-19 antibody (Santa Cruz Biotech). EBC Clone 3.15 and EBC clone 4.12 showed strong reduction of met expression and phospho c-met levels, H441 Clone 3.11 and H441 Clone 3.1 showed intermediate reduction of c-met expression and phospho-met expression, and EBC clone 4.5 showed a smaller reduction of c- met and phospho-c-met expression.

Cell lines EBC clone 4.5, EBC clone 4.12 contained construct shMet4 and cell lines H441 Clone 3.1, H441 Clone 3.11, and EBC Clone 3.15 contained construct shMet 3.

Ligand response experiments. Cells passaged with/without doxycyline for 48 hours (EBC shMet) or 6 days (H441 shMet) were plated at 1×10⁶ cells/well in a 6-well dish with/without DOX (0.1 ug/ml) in 10% FBS-RPMI then incubated overnight at 37 C. Cells were rinsed with PBS, and media was changed to 0.5% BSA-RPMI (with/without doxycyline) to serum starve cells for 2 hours at 37 C. Media containing ligand (20 nM TGFa or 2 nM HRG) was added to wells and incubated for 20 minutes at 37 C. Wells were rinsed with cold TBS then lysed with TBS, 1% NP-40, Complete protease inhibitor cocktail (Roche) and phosphatase inhibitor cocktails 1 and 2 (Sigma). The monolayer and supernatent was scraped from the well and transferred to microfuge tubes where the lysate was incubated on ice for 10-30 minutes. Cell debris was pelleted by microfuge, and the supernatent was transferred to a fresh tube. Protein concentration was quantified by BCA assay (Pierce), and lysates were stored at −20 C until thawed for electrophoresis. 20ug (EBC1) or 15 ug (H441) of whole cell lysate were run on gels and blotted for phospho-c-met (YY1234/35, 3126 from Cell Signaling Technology), total c-met (C12, sc-10 from San Cruz Biotechnology), b-actin (I-19, sc-1616 from Santa Cruz Biotechnology), phospho-EGFR (Y1173, 04-341 from Upstate), total EGFR (MI-12-1, from MBL), phospho-Her2 (YY1121/22, 2243 from Cell Signaling Technology), total Her3 (C18, sc-284, from San Cruz Biotechnology), phospho-Her3 (Y1289, 4791, from Cell Signaling Technology), or total Her3 (C17, sc-285, from Santa Cruz Biotechnology) as described above.

### Results

Retroviruses carrying tetracycline-inducible short-hairpin RNA (shRNA) that target c-met were used to generate stable NSCLC cell line clones that could be induced to express shRNAs to knockdown c-met expression. To examine the effect of c-met knockdown on expression and phosphorylation of EGFR family members in NSCLC cell line EBC1, EBC1 shMet 4.12 cells containing an inducible shRNA directed against met or control shRNA directed against GFP were grown in control media or media containing 0.1 ug/ml Dox (Dox) for 48 hours. After serum-starvation for 2 hours, cells were untreated or treated with TGFα or Heregulin bl for 20 minutes. Whole cell lysates were evaluated for expression of total and phospho-proteins as indicated.

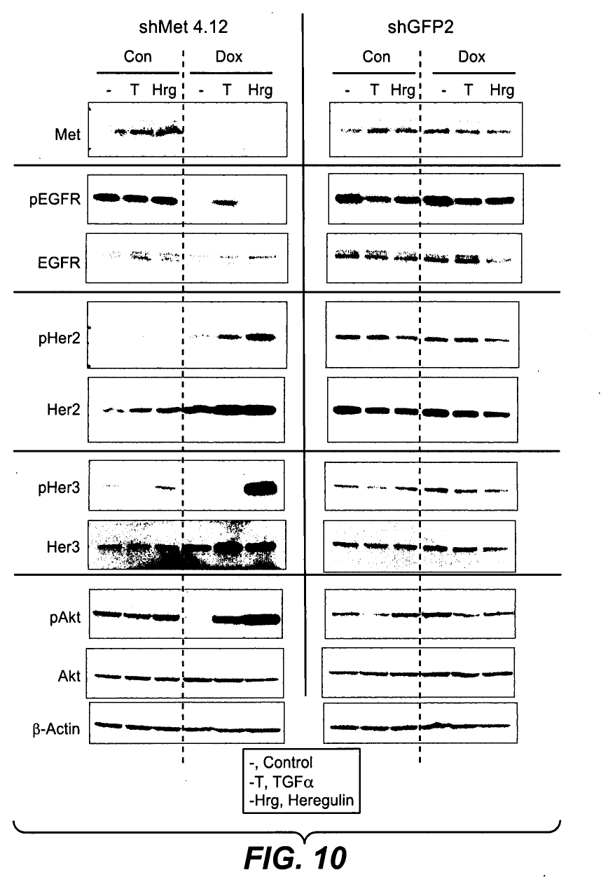

Dox-treated EBCI cells in which c-met protein expression was knocked-down using shRNA (FIG. 10; EBCshMet 4.12, Dox, left panel), but not Dox-treated control EBCI cells (FIG. 10; shGFP2, right panel) showed increased pEGFR and pHer2 in response to TGFa treatment and increased pHer3 in response to Heregulin treatment, as well as increased pAKT with either TGFa or Heregulin treatment. The Dox-treated EBC shMet 4.12 cells (no ligand stimulation) showed increased total Her2 and total Her3, and decreased pEGFR and pHer3. EBC1 cells did not show robust induction of pEGFR, pHer2, pHer3, or pAKT in response to TGFa or Heregulin treatment in the absence of c-met knock-down.

To examine the effect of c-met knockdown on expression and phosphorylation of EGFR family members in another NSCLC cell line, NSCLC H441 cells containing an inducible shRNA directed against met or control shRNA directed against GFP were grown in control media or media containing 0.1 ug/ml Dox (Dox) for 48 hours. After serum-starvation for 2 hours, cells were untreated or treated with TGFα or Heregulin bl for 20 minutes. Whole cell lysates were evaluated for expression of total and phospho-proteins as indicated.

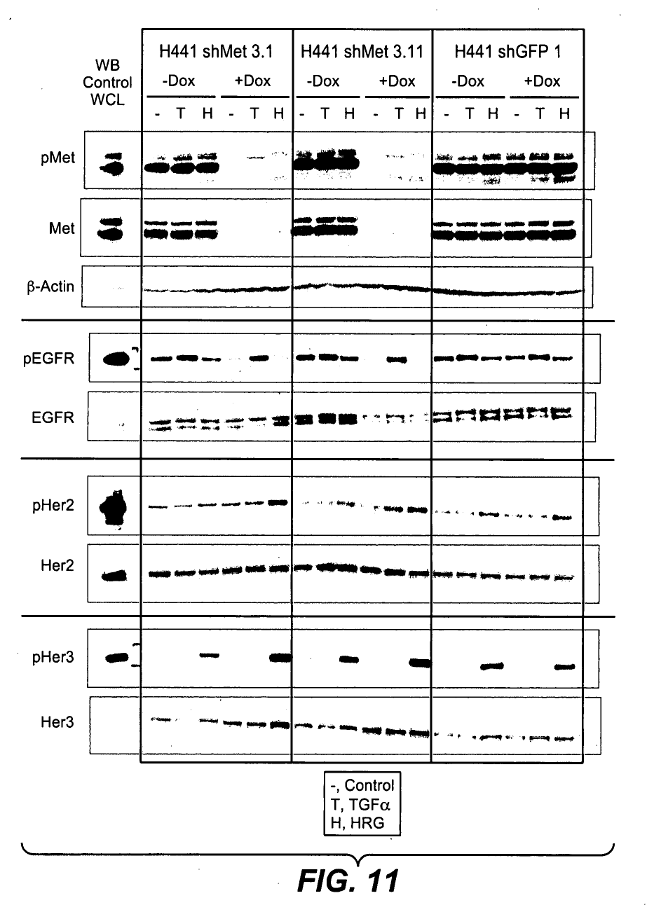

H441 cells in which c-met was knocked-down using shRNA (FIG. 11; Dox-treated shMet 3. 1, left panel and Dox-treated shMet 3.11 middle panel), but not Dox-treated control H441 cells (FIG. 11; shGFP1, right panel) showed enhanced pHer2 and pHer3 in response to Heregulin treatment. The Dox treated shMet 3.1 and shMet 3.11 cells also show increased total Her3 and decrease pEGFR. Unlike EBC1 cells, H441 cells have a slight response to TGFα (PEGFR) and Heregulin (pHer2 and pHer3) without c-met knock-down. EBC1 cells have higher c-met levels than H441 cells. These experiments demonstrated that reduction of c-met expression in NSCLC cell lines leads to decreased basal activation of EGFR (PEGFR) and increased ligand-induced activation of Her 2 and Her3, suggesting that c-met inhibition increases sensitivity to ligands of the HER family of receptors. These results suggest that treatment with the combination of c-met and her2 and/or her3 antagonists will provide significant anti-tumor activity.

### Example 2

### C-Met Activity Regulates HER3 Expression

### Materials and Methods

Western blot analysis of pEGFR and Her3 protein: Cells were plated at 1×10⁶ and incubated 18 hours at 37 C in 10% Tet-approved FBS in RPMI 1640. The next day, media was removed and replaced with fresh normal media, with or without 0.1 ug/ml Dox. 24, 48 and 72 hours after hanging media, proteins were extracted with 1% NP-40/TBS/Roche's Complete protease inhibitor cocktail/Sigma's phosphatase inhibitor cocktails 1 and 2 after a cold TBS rinse. 15 ug of total protein was loaded on Invitrogen's 4-12% Bis-Tris NUPADE gel with MOPS buffer and transferred to PVDF by Invitrogen's iBlot. Membranes were immunoblotted for phosphorylated proteins (PEGFR (Y1173) Upstate 04-341 at a dilution of 1:1000 in 5% BSA/TBST), stripped with Pierce's Restore stripping buffer, then reprobed for total proteins (c-met: SCBT sc-10 at 1:10,000 dilution; Her3: SCBT sc-285 at 1:2000 dilution in 5% nonfat dry milk and TBST). Proteins were detected with Amersham's HRP-conjugated secondary antibodies (Amersham anti-rabbit-HRP, #NA934V; Amersham anti-mouse-HRP) using Amersham's ECL Plus chemiluminescent kit according to the manufacturer's instructions.

Her3 FACS: EBC-1 shMet 4-12 cells were seeded at 106 cells per 10 cm plate in RPMI 1640 (as above) and plates were incubated overnight. Dox was added to plates to a final concentration of 100 ng/ml. Plates were incubated for 48 hrs. Following incubation, cells were trypsinized, centrifuged, then resuspended in cold 200 μL PBS+2%FBS (FACS Buffer) and transferred to 96 well plates. Cells were spun down and resuspended in FACS buffer plus 10 μg/ml of Her3:1638 (3E9.2G6) antibody from Genentech. Cells were incubated for 1 hr on ice, then washed with cold FACS Buffer and resuspended in FACS buffer+1:200 RPE conjugated F(ab′)₂ Goat anti-mouse IgG+IgM (H+L) (Jackson Immuno cat#115-116-068). Cells were incubated on ice for 30 minutes, then washed once with cold FACS buffer and resuspended in FACS buffer plus 7AAD (BD Pharmingen cat#559925). FACS analysis was performed according to the manufacturer's instructions.

Tumor Lysates: EBC-1-shMet xenograft tumors were generated as follows: nude mice (nu/nu) were obtained from Charles River Laboratories (CRL) and were acclimatized for at least one week prior to being put on study. Animals were housed in ventilated caging systems in rooms with filters supplying High-Efficiency Particulate Air (HEPA). Only animals that appeared to be healthy and were free of obvious abnormalities were used for the study.

EBC-1 shMet 4-12 cells were cultured in growth media that consisted of RPMI 1640 media (Invitrogen), 2 mM L-glutamine, and 10% fetal bovine serum. To prepare cells for inoculation into mice, cells were trypsinized, washed with ten milliliters of sterile 1× phosphate buffered saline (PBS). A subset of cells was counted by trypan blue exclusion and the remainder of cells was resuspended in 100 μl of sterile 1×PBS to a concentration of 5×10₇ cells per milliliter. Mice were inoculated subcutaneously in the right sub-scapular region with 5×10₆ EBC-1 cells. Tumors were monitored until they reached a mean volume of 300 mm³. Mice in Group 1 (control group) were switched to drinking water containing 5% sucrose. Mice in Group 2 (c-met knockdown group) were treated with 100 μL MCT, QD, PO, but were switched to drinking water containing 1.0 mg/ML of doxycycline (Dox) in 5% sucrose. All studies and handling of mice complied with the Institutional Animal Care and Use Committee (IACUC) guidelines.

After 3 days of treatment, the mice were sacrificed and the tumors were harvested. Flash frozen EBC-1-shMet-4.12 xenograft tumor samples were placed into 2 mls of cold lysis buffer (PBS+1% TritonX-100+(3×) Phosphatase Cocktail 2 (Sigma cat# P5726)) and Complete Mini EDTA-Free protease inhibitor (Roche #11 836 170 001). Tumors were homogenized with a hand held homogenizer and lysates were incubated on ice for 1 hr with occasional swirling. Lysates were spun down at 10000×G for 10 minutes at 4° C., transferred to a new tube and Her 3 protein was quantified using a BCA assay (Pierce cat#23225).

### Results

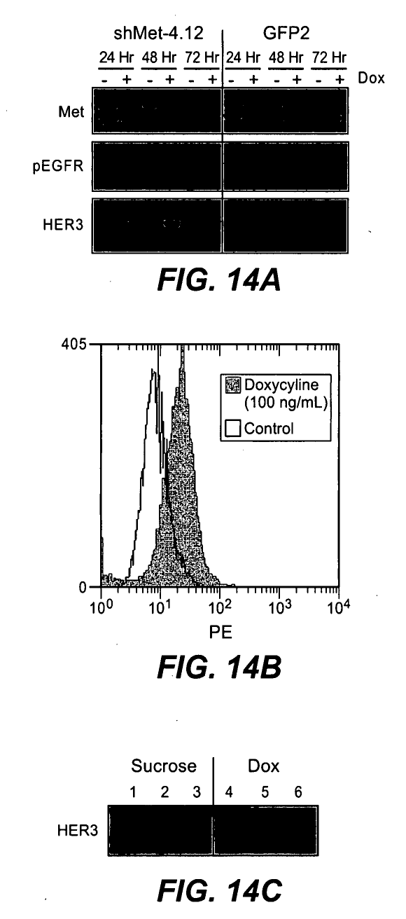

shRNA-mediated knock-down of c-met expression reduced pEGFR levels and significantly increased HER3 protein levels (FIG. 14A). FACS analysis revealed increased surface HER3 levels after c-met knockdown (FIG. 14B). C-met knockdown in EBC-1shMet-4.12 xenograft tumors resulted in an increase in HER3 protein levels (FIG. 14C).

These data demonstrate that c-met activity can regulate HER3 expression level. Specifically, c-met inhibition resulted in increased HER3 protein levels and decreased pEGFR levels. The decrease in pEGFR after c-met inhibition is likely due to decreased autocrine signaling by EGFR ligands and increased HER3 levels might increase erlotinib sensitivity, as has been demonstrated by others (e.g., Yauch et al. Clin Cancer Res (2005) 11 :8686-98). These results suggest that HER3 activity (e.g., signaling through HER2) may increase following inhibition of c-met signaling, and further support the use of combination therapy with c-met and HER3 inhibitors for the treatment of cancer.

### Example 3

### The Combination of C-Met Knockdown and Treatment with HER2 Inhibitor Pertuzumab Significantly Inhibited Tumor Growth

To test whether HER2 dimerization with binding partner HER3 is important in maintaining tumor survival in cell in which c-met function is partially inhibited, EBC-1 shMet4.5-tumor bearing animals were treated with combinations of pertuzumab and Dox.

Materials and Methods

Test material. Pertuzumab (2C4) was provided by Antibody Engineering Department at Genentech, Inc., in a clear liquid form and was diluted in 1×PBS. Control antibodies mouse IgG2a isotype 10D9-1E11-1F12 (anti-Ragweed) antibody and the human IgG1 isotype hu5B6 (anti-gD) antibody were obtained from the Antibody Engineering Department at Genentech, Inc., in a clear liquid form and were diluted in 1×PBS. Doxycycline (Dox) was prepared fresh at 0.5 or 1 mg/mL in 5% sucrose water and was regularly exchanged every 3 days. In Dox studies, control animals were given 5% sucrose water that was exchanged every 3 days. Materials were stored in a refrigerator set to maintain a temperature range of 4° C. to 8° C.

Species. Six-to eight week old nude mice (nu/nu) were obtained from Charles River Laboratories (CRL) and were acclimatized in Genentech's vivarium for at least one week prior to being put on study. Animals were housed in ventilated caging systems in rooms with filters supplying High-Efficiency Particulate Air (HEPA). Only animals that appeared to be healthy and were free of obvious abnormalities were used for the study.

Study design. EBC-1-shMet-4.5 cells were cultured in growth media that consisted of RPMI 1640 media (Invitrogen), 2 mM L-glutamine, and 10% fetal bovine serum. To prepare cells for inoculation into mice, cells were trypsinized, washed with ten milliliters of sterile 1× phosphate buffered saline (PBS). A subset of cells was counted by trypan blue exclusion and the remainder of cells was resuspended in 100 μl of sterile 1× PBS to a concentration of 5×10⁷ cells per milliliter. Mice were inoculated subcutaneously in the shaved right sub-scapular region with 5×10⁶ NCI-H596-cells. Tumors were monitored until they reached a mean volume of 200 mm³.

Mice were randomized into four groups of ten mice each and treatment was initiated (summarized in Table X). Mice in Group 1 were treated with anti-Ragweed control antibody (5 mg/kg, IP, once a week for 5 weeks) and given 5% sucrose drinking water. Mice in Group 2 were treated anti-Ragweed control antibody (5 mg/kg, IP, twice a week for 5 weeks) and given Doxycycline (1 mg/mL) in 5% sucrose drinking water. Mice in Group 3 were treated with Pertuzumab (2C4, at 5 mg/kg, IP, once per week for 4 weeks) and given 5% sucrose drinking water. Mice in Group 4 were treated with Pertuzumab and Doxycycline in 5% sucrose drinking water (as above). Dosing of antibodies was continued for five weeks at which point animals were maintained on Doxycycline treatment, but antibody dosing stopped. Tumor volumes were monitored through day 29.

TABLE 1
Study design

| | | | | | | | Dose |
|---|---|---|---|---|---|---|---|
| | | | | | Dose | Dose Conc. | Volume |
| Group | No./Sex | Test Material | route | Dose Frequency | (mg/kg) | (mg/ml) | (μl) |
| 1 | 10/F | Sucrose; Anti- | Drinking | ad libitum | 5 (anti- | 1.25 (anti- | 100 |
| | | Ragweed | water; | (throughout); | Ragweed) | Ragweed) | |
| | | Control | IP | Once a week × | | | |
| | | Antibody | | 5 weeks | | | |
| 2 | 10/F | Doxycycline; | Drinking | ad libitum | 5 (anti- | 1.25 (anti- | 100 |
| | | Control | water; | (throughout); | Ragweed) | Ragweed); 1 | |
| | | antibody | IP | Once a week × | | (Dox) | |
| | | | | 5 weeks | | | |
| 3 | 10/F | Sucrose; | Drinking | ad libitum | 5 (anti- | 1.25 (anti- | 100 |
| | | Pertuzumab | water; | (throughout); | HER2) | HER2) | |
| | | | IP | Once a week × | | | |
| | | | | 5 weeks | | | |
| 4 | 10/F | Doxycycline; | Drinking | ad libitum | 5 (anti- | 1.25 (anti- | 100 |
| | | Pertuzumab | water; | (throughout); | HER2) | HER2); 1 | |
| | | | IP | Once a week × | | (Dox) | |
| | | | | 5 weeks | | | |

For all studies reported here, animals were taken off study if tumors reached greater than 2000 mm³ or tumors showed signs of necrotic lesions. If more than 50% of the animals had to be taken off study from any given group, treatment in that group was halted and all animals were taken off study. All studies and handling of mice complied with the Institutional Animal Care and Use Committee (IACUC) guidelines.

Tumor and Body Weight Measurement. Tumor volumes were measure in two dimensions (length and width) using UltraCal-IV calipers (Model 54-10-111, Fred V. Fowler Company, Inc.; Newton, Mass.). The following formula was used with Excel v11.2 (Microsoft Corporation; Redmond, Wash.) to calculate tumor volume:

Tumor Volume (mm³)=(length·width²)·0.5

Efficacy Data Analysis. Tumor inhibition was plotted using KaleidaGraph 3.6 (Synergy Software; Reading, Pa.). Percent growth inhibition (% Inh) was calculated as follows:

% Ihn=100×(1−[Tumor Size (Treated)/Tumor Size (Vehicle)])

Tumor incidence was determined by the number of measurable tumors in each group at the end of study. Partial regression (PR) is defined as tumor regression of >50% but <100% of starting tumor volume at any day during the study. Complete regression (CR) is defined as tumor regression of 100% from initial starting tumor volume at any day during the study.

Mean tumor volume and standard error of the mean (SEM) were calculated using JMP software, version 5.1.2 (SAS Institute; Cary, N.C.). Data analysis and generation of p-values using either Student's t-test or the Dunnett's t-test was also done using JMP software, version 5.1.2.

### Results

Stable EBC-1 clones that could be induced to express shRNAs to knockdown c-met expression were generated using retroviruses carrying a tetracycline-inducible short-hairpin RNA (shRNA) targeting c-met. The EBC-1 non-small cell lung cancer (NSCLC) cell line is highly amplified for c-met and expresses high amounts of the c-met receptor which acts in a ligand-independent manner to drive cell and tumor growth. The EBC-1 shMet-4.5 clone displayed partial knocked-down of c-met expression following induction of shRNA expression with Dox. Reduction in c-met expression also resulted in effects upon cell growth and survival in this clone: induction of shRNA expression decreased cell number when assayed in in vitro cell viability assays, and induction of shRNA expression after tumor formation in a xenograft model inhibited tumor growth but did not cause tumor regression. Clone shMet-4.5 was selected for use in experiments evaluating the effect of combining knock-down of c-met expression with pertuzumab treatment, as described below.

To test whether HER2 dimerization with binding partner HER3 is important in maintaining tumor survival in cell in which c-met function is partially inhibited, EBC-1 shMet4.5-tumor bearing animals were treated with combinations of pertuzumab and Dox.

The EBC-1 shMet-4.5 NSCLC cell line was inoculated into nude mice and then animals were monitored for tumor growth until the engrafted cells had formed tumors of about 200 mm³. Mice were then grouped into four treatment arms; Group 1: Control, Group 2: Doxycycline (Dox), Group 3: pertuzumab (1.25 mg/kg), and Group 4: pertuzumab+Dox (See Table 1).

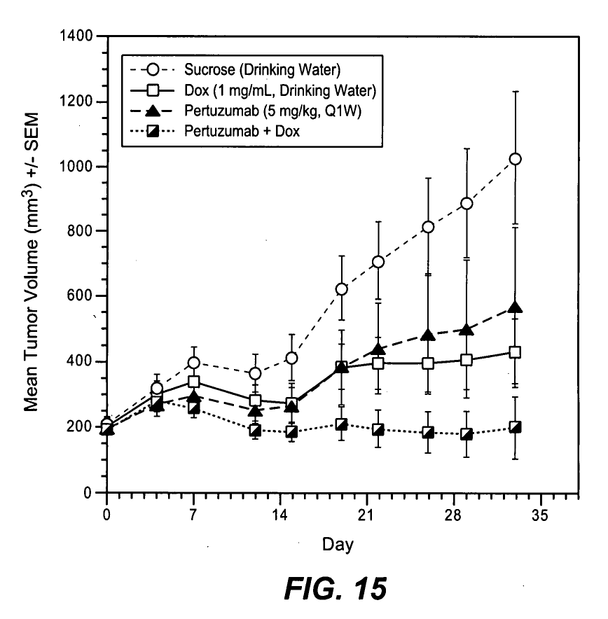

Knockdown of c-met gene expression by treatment with Dox in the drinking water resulted in a decrease in total Met protein levels ultimately leading to a significant, but incomplete inhibition of tumor growth compared to sucrose control at day 29 (54% tumor inhibition; Student's t-test, ρ=0.011) (FIG. 15). Treatment with pertuzumab alone resulted in 43.6% tumor inhibition, however this treatment did not reach significance compared to the sucrose control at day 29 (Student's t-test, ρ=0.084).

Treatment with the combination of pertuzumab and Dox resulted in a significant improvement in efficacy, resulting in a 79.4% reduction in tumor growth compared to sucrose control at day 29 (Student's t-test, p=0.001; FIG. 15). Pertuzumab and Dox-treated tumors remained static over the 4-5 week experimental period. The increase in efficacy observed in the pertuxumab plus Dox treatment arm was significantly better than that observed with Dox treatment alone (Student's t-test, p=0.03); however combination treatment with pertuzumab and Dox narrowly missed significance when compared with treatment with Pertuzumab alone at day 29 (Student's t-test, p=0.083), due to the high variability observed in the response to single agent pertuzumab.

These results show that inhibition of c-met and inhibition of Her2 dimerization in the EBC-1 shMet-4.5 xenograft model resulted in a significant reduction in tumor growth. Thus, tumors in which c-met expression and activity are partially inhibited utilize the HER pathway to ensure tumor growth and survival. This indicates that HER plays a role in tumor survival and growth in tumors in which c-met is inhibited.

### PARTIAL LIST OF REFERENCES

Bhargava, M, Joseph, A, Knesel, J, Halaban, R, Li, Y, Pang, S, Goldberg, I, Setter, E, Donovan, M A, Zamegar, R, Michalopoulos, G A, Nakamura, T, Faletto, D. and Rosen, E. (1992). Scatter factor and hepatocyte growth factor: activities, properties, and mechanism. Cell Growth Differ., 3(1); 11-20.

Kong-Beltran, M., Seshagiri, S., Zha, J., Zhu, W., Bhawe, K., Mendoza, N., Holcomb, T., Pujara, K., Stinson, J., Fu, L., Severin, C., Rangell, L., Schwall, R., Amler,L., Wickramasinghe, D., Yauch, R. (2006). Somatic Mutations Lead to an Oncogenic Deletion of Met in Lung Cancer. Cancer Res, 66 (1); 283-289.

Peschard, P., Foumier, T. M., Lamorte, L, Naujokas, M. A., Band, H., Langton, W. Y., Park, M. (2001). Mutation of the c-Cbl TKB domain binding site on the Met receptor tyrosine kinase converts it into a transforming protein. Mol Cell., 8(5); 995-1004.

Ridgeway, J. B. B., Presta, L. G., Carter, P. (1996). ‘Knobs-into-holes’ engineering of antibody CH3 domains for heavy chain heterodimerization. Protein Engin. 9 (7): 617-621.

Rong, S., Bodescot, M., Blair, D., Dunn, J., Nakamura, T., Mizuno, K., Park, M., Chan, A., Aaronson, S., Vande Woude, G. F. (1992). Tumorigenicity of the met proto-oncogene and the gene for the hepatocyte growth factor. Mol Cell Biol., 12(11); 5152-5158.

Zhang, Y-W., Su, Y., Lanning, N., Gustafson, M., Shinomiya, N., Zhao, P., Cao, B., Tsarfaty, G., Wang, L-M, Hay, R., Vande Woude, G. F. (2005). Enhanced growth of human met-expressing xenografts in a new strain of immunocompromised mice transgenic for human hepatocyte growth factor/scatter factor. Oncogene, 24; 101-106.

Although the foregoing invention has been described in some detail by way of illustration and example for purposes of clarity of understanding, the descriptions and examples should not be construed as limiting the scope of the invention.

What is claimed is:

1. A method of treating cancer in a subject, comprising administering to the subject a therapeutically effective amount of a c-met antagonist and an HER antagonist.

2. The method of claim 1, wherein the c-met antagonist is an antibody.

3. The method of claim 2, wherein the antibody is a monovalent antibody.

4. The method of claim 3, wherein the antibody is monovalent and further comprises a Fc region, wherein the Fc region comprises a first and a second polypeptide, wherein the first polypeptide comprises the Fc sequence depicted in FIG. 12 (SEQ ID NO: 33) and the second polypeptide comprises the Fc sequence depicted in FIG. 13 (SEQ ID NO: 34).

5. The method of claim 3, wherein the antibody comprises (a) a first polypeptide comprising a heavy chain variable domain having the sequence:
QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYWSPLDYWGQGTSVTVSS (SEQ ID NO:35), CH1 sequence depicted in FIG. 12 (SEQ ID NO: 32), and the Fc sequence depicted in FIG. 12 (SEQ ID NO: 33); and (b) a second polypeptide comprising a light chain variable domain having the sequence:
DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ ID NO:36), and CL1 sequence depicted in FIG. 12 (SEQ ID NO: 24); and (c) a third polypeptide comprising the Fc sequence depicted in FIG. 13 (SEQ ID NO: 34).

6. The method of claim 1, wherein the HER antagonist is a HER2 antagonist.

7. The method of claim 1, wherein the HER antagonist is a HER3 antagonist.

8. The method of claim 1, wherein the HER antagonist is an antibody.

9. The method of claim 8, wherein the antibody is an anti-HER2 antibody.

10. The method of claim 8, wherein the antibody is an anti-HER3 antibody.

11. The method of claim 1, wherein the HER antagonist is a HER dimerization inhibitor.

12. The method of claim 1, wherein the cancer is selected from the group consisting of breast cancer, colorectal cancer, rectal cancer, non-small cell lung cancer, non-Hodgkins lymphoma, renal cell cancer, prostate cancer, liver cancer, pancreatic cancer, soft-tissue sarcoma, kaposi's sarcoma, carcinoid carcinoma, head and neck cancer, gastric cancer, melanoma, ovarian cancer, mesothelioma, and multiple myeloma

13. The method of claim 10, wherein the cancer is non-small cell lung cancer.

14. The method of claim 1, further comprising administering to the subject a chemotherapeutic agent.


SEQUENCE LISTING (52 sequences)

SEQ ID NO: 1 - protein, 107 aa, Artificial Sequence
  Description of Artificial Sequence Synthetic polypeptide
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 2 - protein, 119 aa, Artificial Sequence
  Description of Artificial Sequence Synthetic polypeptide
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 3 - protein, 107 aa, Artificial Sequence [sequence omitted]

SEQ ID NO: 4 - protein, 119 aa, Artificial Sequence [sequence omitted]

SEQ ID NO: 5 - protein, 107 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 6 - protein, 119 aa, Homo sapiens
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 7 - protein, 23 aa, Artificial Sequence
  Description of Artificial Sequence Synthetic peptide
DIQMTQSPSSLSASVGDRVTITC

SEQ ID NO: 8 - protein, 15 aa, Artificial Sequence
  Description of Artificial Sequence Synthetic peptide
WYQQKPGKAPKLLIY

SEQ ID NO: 9 - protein, 32 aa, Artificial Sequence
  Description of Artificial Sequence Synthetic polypeptide
GVPSRFSGSGSGTDFTLTISSLQPEDFATYYC

SEQ ID NO: 10 - protein, 11 aa, Artificial Sequence
  Description of Artificial Sequence Synthetic peptide
FGQGTKVEIKR

SEQ ID NO: 11 - protein, 17 aa, Artificial Sequence
  Description of Artificial Sequence Synthetic peptide
KSSQSLLYTSSQKNYLA

SEQ ID NO: 12 - protein, 7 aa, Artificial Sequence
  Description of Artificial Sequence Synthetic peptide
WASTRES

SEQ ID NO: 13 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 233 aa, Artificial Sequence
  Description of Artificial Sequence Synthetic polypeptide
MGWSCIILFLVATATGVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 467 aa, Artificial Sequence
  Description of Artificial Sequence Synthetic polypeptide
MGWSCIILFLVATATGVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 20 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 21 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 22 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 23 - protein, 9 aa, Artificial Sequence
  Description of Artificial Sequence Synthetic peptide
QQYYAYPWT

SEQ ID NO: 24 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 25 aa, Artificial Sequence [sequence omitted]

SEQ ID NO: 26 - protein, 13 aa, Artificial Sequence
  Description of Artificial Sequence Synthetic peptide
WVRQAPGKGLEWV

SEQ ID NO: 27 - protein, 30 aa, Artificial Sequence
  Description of Artificial Sequence Synthetic polypeptide
RFTISADTSKNTAYLQMNSLRAEDTAVYYC

SEQ ID NO: 28 - protein, 11 aa, Artificial Sequence
  Description of Artificial Sequence Synthetic peptide
WGQGTLVTVSS

SEQ ID NO: 29 - protein, 10 aa, Artificial Sequence
  Description of Artificial Sequence Synthetic peptide
GYTFTSYWLH

SEQ ID NO: 30 - protein, 18 aa, Artificial Sequence
  Description of Artificial Sequence Synthetic peptide
GMIDPSNSDTRFNPNFKD

SEQ ID NO: 31 - protein, 12 aa, Artificial Sequence
  Description of Artificial Sequence Synthetic peptide
ATYRSYVTPLDY

SEQ ID NO: 32 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 33 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 119 aa, Artificial Sequence
  Description of Artificial Sequence Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSS

SEQ ID NO: 36 - protein, 113 aa, Artificial Sequence
  Description of Artificial Sequence Synthetic polypeptide
DIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIK

SEQ ID NO: 37 - protein, 11 aa, Artificial Sequence
  Description of Artificial Sequence Synthetic peptide
DICLPRWGCLW

SEQ ID NO: 38 - protein, 5 aa, Artificial Sequence
  Description of Artificial Sequence Synthetic peptide
LDAQT

SEQ ID NO: 39 - protein, 9 aa, Artificial Sequence
  Description of Artificial Sequence Synthetic peptide
LTEKRKKRS

SEQ ID NO: 40 - protein, 8 aa, Artificial Sequence
  Description of Artificial Sequence Synthetic peptide
KPDSAEPM

SEQ ID NO: 41 - protein, 8 aa, Artificial Sequence
  Description of Artificial Sequence Synthetic peptide
NVRCLQHF

SEQ ID NO: 42 - protein, 10 aa, Artificial Sequence
  Description of Artificial Sequence Synthetic peptide
GFTFTDYTMX

SEQ ID NO: 43 - protein, 17 aa, Artificial Sequence
  Description of Artificial Sequence Synthetic peptide
DVNPNSGGSIYNQRFKG

SEQ ID NO: 44 - protein, 10 aa, Artificial Sequence
  Description of Artificial Sequence Synthetic peptide
NLGPSFYFDY

SEQ ID NO: 45 - protein, 11 aa, Artificial Sequence
  Description of Artificial Sequence Synthetic peptide
KASQDVSIGVA

SEQ ID NO: 46 - protein, 7 aa, Artificial Sequence
  Description of Artificial Sequence Synthetic peptide
SASYXXX

SEQ ID NO: 47 - protein, 9 aa, Artificial Sequence
  Description of Artificial Sequence Synthetic peptide
QQYYIYPYT

SEQ ID NO: 48 - DNA, 65 bp, Artificial Sequence
  Description of Artificial Sequence Synthetic oligonucleotide
gatccccgaacagaatcactgacatattcaagagatatgtcagtgattctgttcttttttggaaa

SEQ ID NO: 49 - DNA, 65 bp, Artificial Sequence
  Description of Artificial Sequence Synthetic oligonucleotide
gatccccgaaactgtatgctggatgattcaagagatcatccagcatacagtttcttttttggaaa

SEQ ID NO: 50 - DNA, 67 bp, Artificial Sequence
  Description of Artificial Sequence Synthetic oligonucleotide
gatccccagatccgccacaacatcgattcaagagatcgatgttgtggcggatcttgttttttggaaa

SEQ ID NO: 51 - protein, 217 aa, Artificial Sequence
  Description of Artificial Sequence Synthetic polypeptide
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK